(19) United States
(12) Patent Application Publication
(10) Pub. No.: US 2026/0042871 A1
(43) Pub. Date: Feb. 12, 2026

(54) CHOLESTEROL-MODIFIED HYALURONIC ACIDS

(71) Applicant: UNIVERSITY OF MASSACHUSETTS, Boston, MA (US)

(72) Inventors: Jie SONG, Shrewsbury, MA (US); Shing-yun CHANG, Westborough, MA (US)

(21) Appl. No.: 19/100,683

(22) PCT Filed: Aug. 4, 2023
(86) PCT No.: PCT/US2023/029566

§ 371 Date: Feb. 3, 2025

Related U.S. Application Data
(60) Provisional application No. 63/395,222, filed on Aug. 4, 2022.

(51) Int. Cl. C08B 37/08; A61K 31/56; A61K 31/7088; A61K 31/74; A61K 33/24; A61K 35/00; A61K 38/16; A61K 45/06; A61K 47/36; A61K 49/00; C08J 3/24

(52) U.S. Cl. CPC C08B 37/0072; A61K 31/56; A61K 31/7088; A61K 31/74; A61K 33/24; A61K 35/00; A61K 38/16; A61K 45/06; A61K 47/36; A61K 49/00; C08J 3/24

(57) ABSTRACT

Disclosed herein are cholesterol-modified hyaluronic acid polymers, as well as methods of making and using thereof.

### CROSS REFERENCE TO RELATED APPLICATIONS

This application claims benefit of U.S. Provisional Application No. 63/395,222, filed Aug. 4, 2022, which is hereby incorporated by reference in its entirety.

### BACKGROUND

Hydrogels have broad utility in tissue engineering, cell and biotherapeutics delivery and guide tissue regeneration. The importance of viscoelasticity of cell-laden hydrogel in dictating the fate (e.g., differentiation of stem cells) and function (e.g., cell-specific matrix deposition) of encapsulated cells has been addressed in the context of synthetic hydrogels in recent years. Of note, reversible covalent cross-linking in hydrogels was implemented as a way to modulate viscoelasticity to facilitate the growth and expansion of cells. Chemical cross-linking with short lifetime explored as reversible cross-linking also included, for example, Diels-Alder adducts, disulfide and oxime bonds. Meanwhile, various physical cross-linking was used to modulate viscoelasticity to prepare cell-laden hydrogels where the dynamic ionic crosslinking or π-π stacking/hydrophobic interaction based physical crosslinking were exploited to influence encapsulated cell behavior. A common challenge shared by current methods is that the magnitude of stoichiometrically controlled tuning of hydrogel viscoelasticity is limited.

Polyethylene glycol (PEG) is a synthetic polymer quite frequently used as drug carriers (including in vaccine formulations) and hydrogels in regenerative medicine due to its perceived biocompatibility, although it is increasingly recognized for its ability to still elicit negative immune responses (allergic reactions) in a subset of population. In addition, the low-fouling nature of PEG hydrogels also prevents some encapsulated cells such as mesenchymal stem cells from properly adhering and proliferating without explicit chemical functionalization (e.g., via covalent attachment of integrin binding RGD peptide). Consequently, native biopolymer-derived hydrogels or nanoparticles/microparticles, such as gelatin and hyaluronic acid, have been actively explored as alternatives for cell and biotherapeutics delivery and regenerative medicine applications.

Hyaluronic acid (HA), as one of the major components in extracellular and pericellular matrices of multiple tissues including cartilage, is of particular interest due to its cyto- and bio-compatibility, specific interaction with cell surface receptors, biodegradability, low-immunogenicity and versatile viscoelasticity driven by wide-ranging molecular weights.

Thus, it has been widely used for tissue engineering and drug delivery. However, to formulate highly bio-degradable and linear HA into a free-standing gel, nanogel, nanoparticles, or microparticles, proper crosslinking is needed.

The most popular method for crosslinking HA has evolved from photo-crosslinking (which is known for negatively impact the encapsulated cells due to potential UV damage) to catalyst-free and irradiation-free, strain-promoted alkyne-azide cycloaddition (SPAAC) that can be carried out under physiological conditions. These covalent crosslinks do not provide means to tune viscoelasticity.

Accordingly, improved HA compositions are needed for a wide variety of applications.

### SUMMARY

Described herein are covalently-modified hyaluronic acid polymers. The covalently-modified hyaluronic acid polymers can be functionalized, for example via click chemistry, to include one or more cholesterol moieties. Optionally, the covalently-modified hyaluronic acid polymers can be further covalently crosslinked (e.g., using a bifunctional or polyfunctional crosslinker which covalently crosslinks the covalently-modified hyaluronic acid polymers via click reactions). These modified hyaluronic acid polymers can prepare particles (e.g., nanoparticles and/or microparticles) and 3D viscoelastic hydrogels.

For example, provided herein are modified hyaluronic acid polymers comprising one or more covalently modified monomers defined by Formula I below

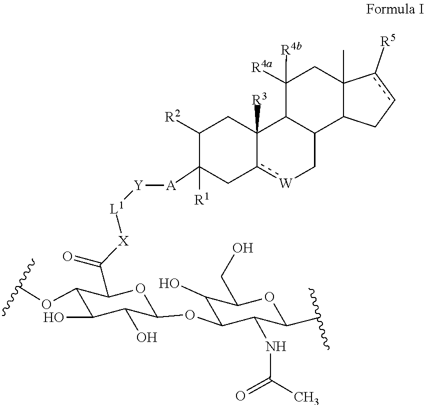

wherein
[symbol] denotes carbon-carbon bond or carbon-carbon double bond;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is NH, O, or S;
R^{a} is

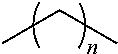

wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is

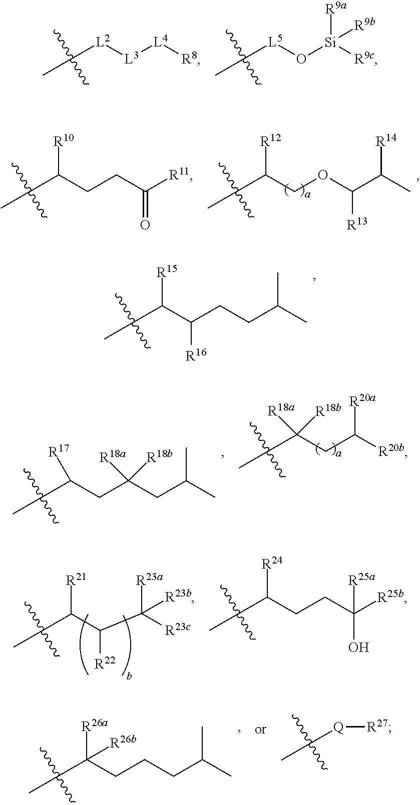

L² is absent,

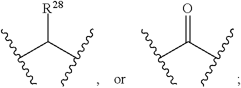

R²⁸ is absent or C₁-C₆ alkyl;
L³ is absent, or

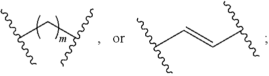

m is an integer 1, 2, or 3;
L⁴ is absent,

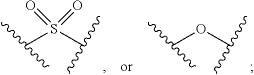

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or

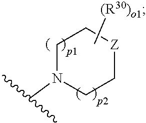

R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form

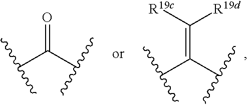

were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

In some aspects, the modified hyaluronic acid can comprise a random copolymer defined by Formula II below

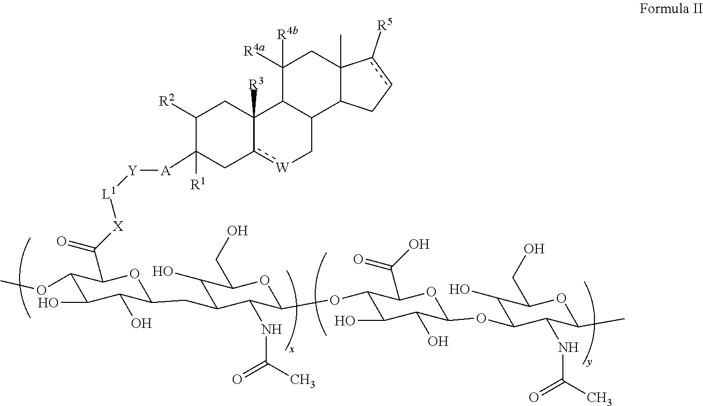

wherein
x and y are each independently integers from 1 to 2500, wherein x+y is no more than 2500, and wherein x and y represent the relative portion of each monomer within the random copolymer;
[symbol] denotes carbon-carbon bond or carbon-carbon double bond;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is NH, O, or S;
R^{a} is

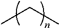

wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is

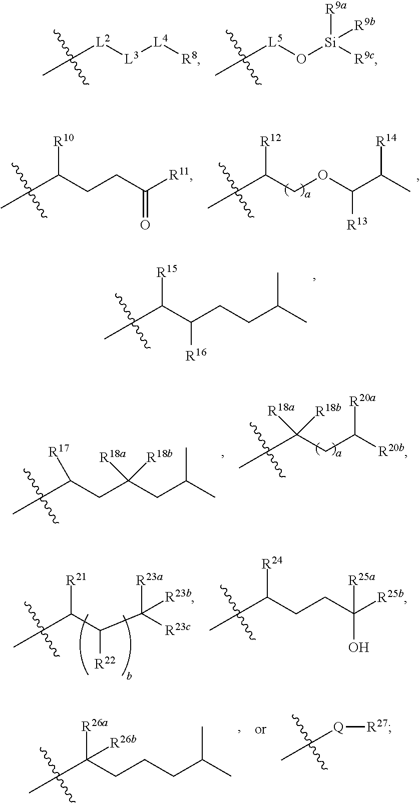

L² is absent,

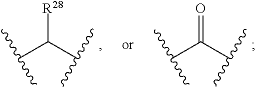

R²⁸ is absent or C₁-C₆ alkyl;
L³ is absent, or

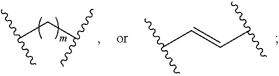

m is an integer 1, 2, or 3;
L⁴ is absent,

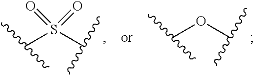

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or

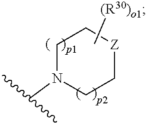

R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form

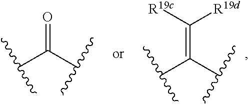

were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3,
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

In some aspects, the modified hyaluronic acid can be covalently crosslinked. In certain aspects, the modified hyaluronic acid can be covalently crosslinked using click chemistry.

In some aspects, the modified hyaluronic acid can comprise a random copolymer defined by Formula III below

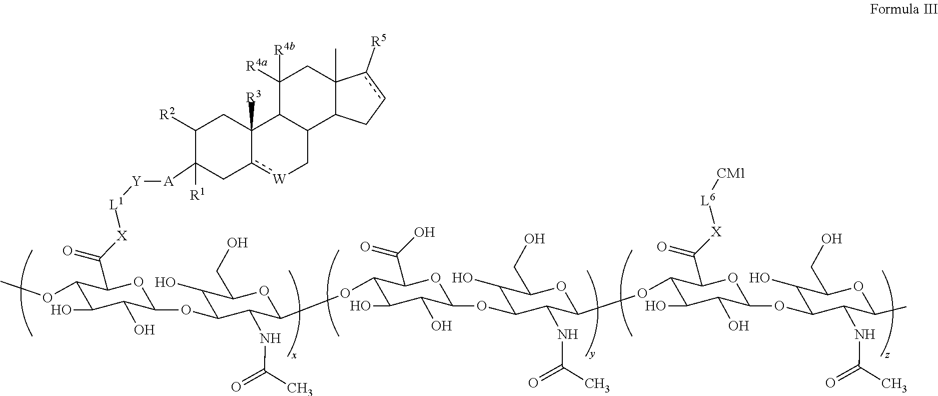

wherein
x, y, and z are each independently integers from 1 to 2500, wherein x, y, and z represent the relative portion of each monomer within the random copolymer, and wherein x+y+z is no more than 2500;
[symbol] denotes carbon-carbon bond or carbon-carbon double bond;
L⁶ is absent, or represents a linking group;
CM1 represents a first click motif;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two click motifs;
X is, independently for each occurrence, NH, O, or S;
R^{a} is

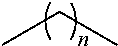

wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is

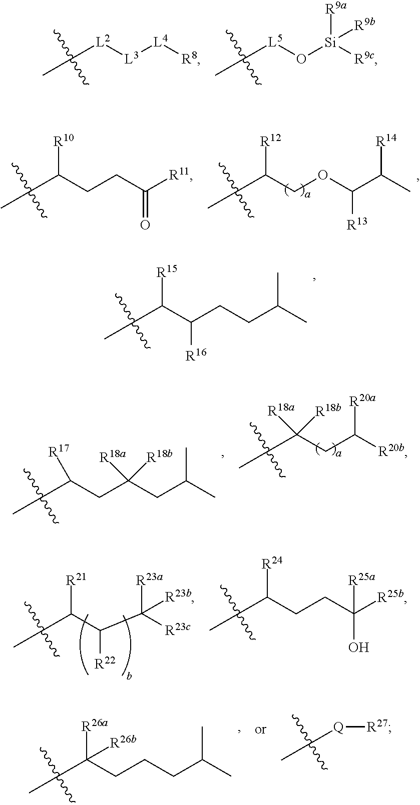

L² is absent,

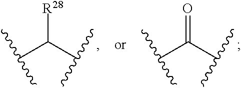

R²⁸ is absent or C₁-C₆ alkyl;
L³ is absent, or

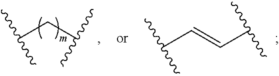

m is an integer 1, 2, or 3;
L⁴ is absent

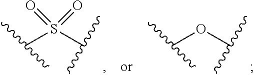

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or

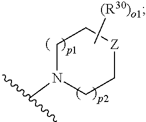

R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form

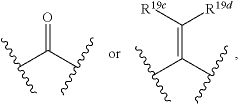

were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

In some aspects, CM1 can comprise an azide.

In some aspects, the modified hyaluronic acid can be covalently crosslinked by reaction of the copolymer defined by Formula III with a crosslinker defined by the structure below

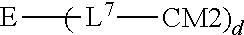

wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

In some aspects, the click reaction can comprise a strain-promoted alkyne-azide cycloaddition (SPAAC). In some aspects, CM2 can comprise an alkyne. In other aspects, the CM2 can comprise a dibenzocyclooctyne (DBCO) moiety.

In some aspects, E can comprise an oligomer or polymer.

In some aspects of Formula I, Formula II, and Formula III, R⁵ can be

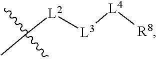

wherein
L² is absent,

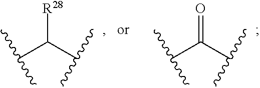

R²⁸ is absent or C₁-C₆ alkyl;
L³ is absent, or

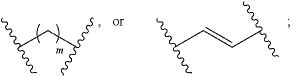

m is an integer 1, 2, or 3;
L⁴ is absent, or

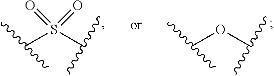

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl.

In some aspects of Formula I, Formula II, and Formula III, R¹ is —CH₃.

In some aspects of Formula I, Formula II, and Formula III, A is O.

In some aspects of Formula I, Formula II, and Formula III, L² is

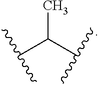 [SMILES: *C(*)C]

In some aspects of Formula I, Formula II, and Formula III, L³ is

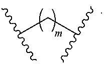

In some aspects of Formula I, Formula II, and Formula III, m is 3.

In some aspects of Formula I, Formula II, and Formula III, L⁴ is absent.

In some aspects of Formula I, Formula II, and Formula III, R⁸ is methyl, ethyl, n-propyl, isopropyl, n-butyl, isobutyl, t-butyl, pentyl, hexyl, heptyl, octyl, nonyl, or decyl. In certain aspects of Formula I, Formula II, and Formula III, R⁸ is isopropyl.

In some aspects, the modified hyaluronic acid can comprise one or more covalently modified monomers defined by Formula IA

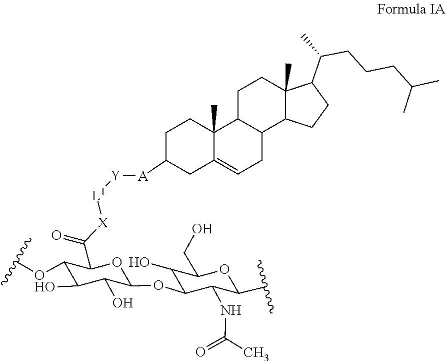

wherein
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR⁴C(═O)—;
L¹ is a linking group comprising a moiety formed by the chemical reaction of two click motifs;
X is NH, O, or S; and
R^{a} is

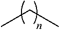

an integer from 0 to 12.

In some aspects, the modified hyaluronic acid can comprise a random copolymer defined by Formula IIA below

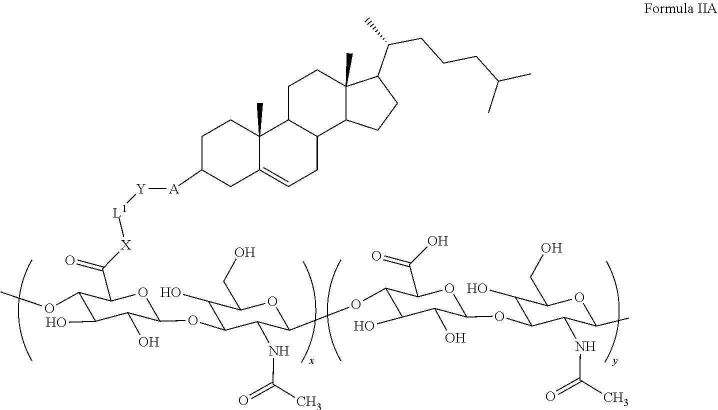

wherein
x and y are each independently integers from 1 to 2500, wherein x and y represent the relative portion of each monomer within the random copolymer, and wherein x+y+z is no greater than 2500;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is NH, O, or S; and
R^{a} is

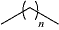

wherein n is an integer from 0 to 12.

In some aspects, the modified hyaluronic acid can comprise a random copolymer defined by Formula IIIA below

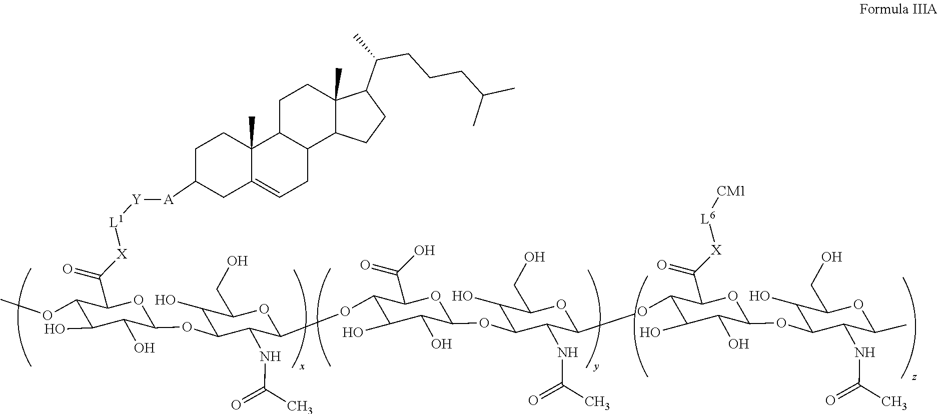

wherein
x, y, and z are each independently integers from 1 to 2500, wherein x, y, and z represent the relative portion of each monomer within the random copolymer, and wherein x+y+z is no greater than 2500;
L⁶ is absent, or represents a linking group;
CM1 represents a first click motif;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two click motifs;
X is, independently for each occurrence, NH, O, or S;
R^{a} is

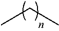

wherein n is an integer from 0 to 12.

In some aspects, CM1 can comprise an azide.

In some aspects, the modified hyaluronic acid can be covalently crosslinked by reaction of the copolymer defined by Formula IIIA with a crosslinker defined by the structure below

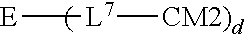

wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

In some aspects, the click reaction can comprise a strain-promoted alkyne-azide cycloaddition (SPAAC). In some aspects, CM2 can comprise an alkyne. In other aspects, the CM2 can comprise a dibenzocyclooctyne (DBCO) moiety.

In some aspects, E can comprise an oligomer or polymer.

In some aspects of Formula I, Formula IA. Formula II, Formula IIA, Formula III, and Formula IIIA, X is NH.

In some aspects of Formula I, Formula IA, Formula II, Formula IIA, Formula III, and Formula IIIA, Y is —NR^{a}C(═O)—.

In some aspects of Formula I, Formula IA, Formula II, Formula IIA, Formula III, and Formula IIIA, L¹ comprises a moiety formed by a strain-promoted alkyne-azide cycloaddition (SPAAC). In certain aspects of Formula I, Formula IA, Formula II, Formula IIA, Formula III, and Formula IIIA, L¹ comprises a moiety formed by reaction of an azide moiety with a dibenzocyclooctyne (DBCO) moiety.

In some aspects of Formula I, Formula IA. Formula II, Formula IIA, Formula III, and Formula IIIA, L¹ further comprises an oligomer or polymer.

Also provided are modified hyaluronic acid polymers that comprise one or more covalently modified monomers defined by Formula IV

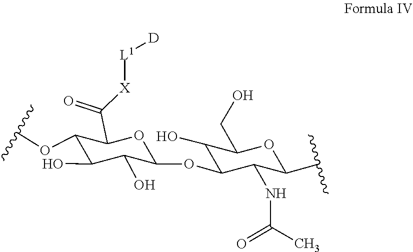

wherein
L¹ is a linking group comprising a moiety formed by the chemical reaction of two dick motifs;
X is, independently for each occurrence, NH, O, or S;
D represents an oligomer or copolymer (block copolymer or random copolymer) defined by the formula below

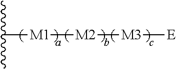

wherein M1 comprises a repeat unit comprising a hydrophilic sidechain;
wherein M2 comprises a repeat unit comprises a cholesterol-containing sidechain;
wherein M3 comprises a repeat unit comprising a hydrophilic sidechain;
a is an integer from 0 to 500;
b is an integer from 2 to 500;
c is an integer from 0 to 500; and
E is absent, or represents a terminal unit optionally selected from the group consisting of dithioester, trithiocarbonate, xanthate.

In some aspects, M1, M2, and M3 are independently repeat units obtainable by polymerization of a (meth)acrylate or (meth)acrylamide monomer, such as reversible addition-fragmentation chain-transfer polymerization (RAFT) of a (meth)acrylate or (meth)acrylamide monomer.

In some aspects, at least one of a and c is from 2 to 500.

In some aspects, M1 and M3 are independently repeat units obtainable by polymerization of a (meth)acrylate or (meth)acrylamide monomer comprising a hydrophilic oligomer or polymer sidechain (e.g., an oligo- or polyethylene glycol sidechain), such as reversible addition-fragmentation chain-transfer polymerization (RAFT) of a (meth)acrylate or (meth)acrylamide monomer comprising a hydrophilic oligomer or polymer sidechain, such as an oligo- or polyethylene glycol sidechain.

In some aspects, M2 is a repeat unit obtainable by polymerization of a (meth)acrylate or (meth)acrylamide monomer comprising a (meth)acrylate or (meth)acrylamide monomer comprising a sidechain defined by the formula below, such as reversible addition-fragmentation chain-transfer polymerization (RAFT) of a (meth)acrylate or (meth)acrylamide monomer comprising a sidechain defined by the formula below

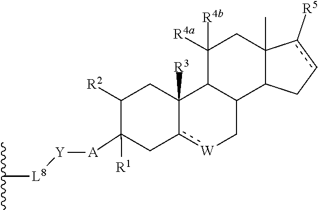

A is absent, or is O or S;
Y is absent, or represents —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
[symbol] denotes carbon-carbon bond or carbon-carbon double bond;
L⁸ is absent, or is a linking group;
R^{a} is

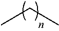

wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is

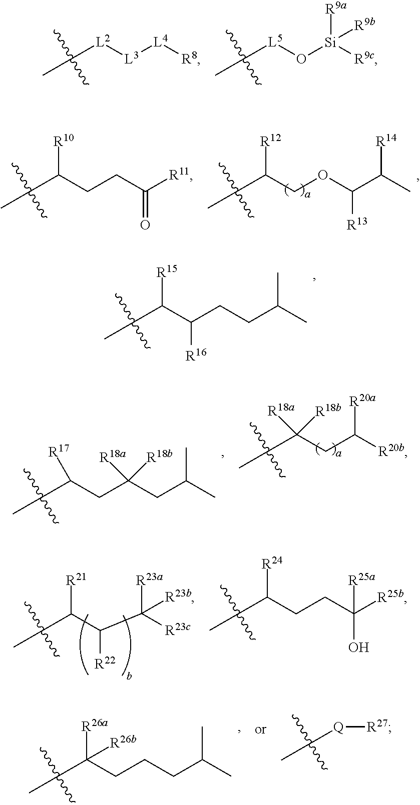

L² is absent,

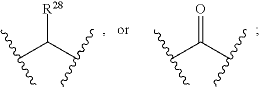

R²⁸ is absent or C₁-C₆ alkyl;
L³ is absent, or

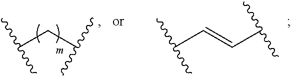

m is an integer 1, 2, or 3;
L⁴ is absent,

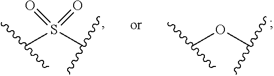

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or

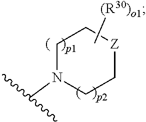

R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form

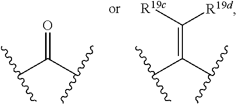

were R^{19a} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

In some aspects, M2 is a repeat unit obtainable by polymerization of a (meth)acrylate or (meth)acrylamide monomer comprising a (meth)acrylate or (meth)acrylamide monomer comprising a sidechain defined by the formula below, such as reversible addition-fragmentation chain-transfer polymerization (RAFT) of a (meth)acrylate or (meth)acrylamide monomer comprising a sidechain defined by the formula below

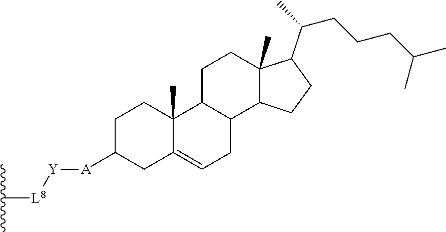

A is absent, or is O or S;
Y is absent, or represents —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; and
L⁸ is absent, or is a linking group.

In some aspects, the modified hyaluronic acid can comprise a random copolymer defined by Formula IVA below

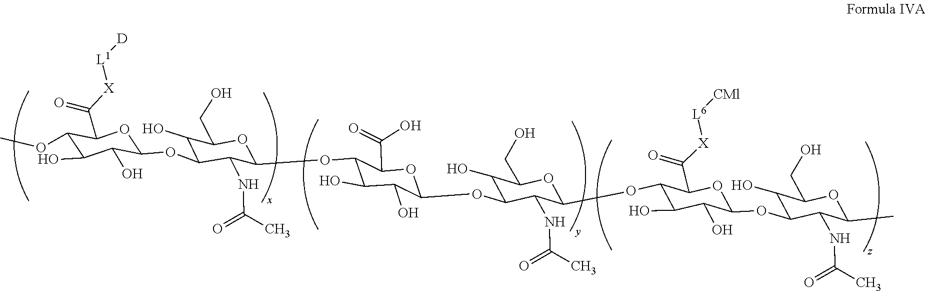

wherein
x, y, and z are each independently integers from 1 to 2500, wherein x, y, and z represent the relative portion of each monomer within the random copolymer, and wherein x+y+z is no greater than 2500;
L¹ is a linking group comprising a moiety formed by the chemical reaction of two click motifs;
X is, independently for each occurrence, NH, O, or S;
D represents an oligomer or copolymer (block copolymer or random copolymer) defined by the formula below

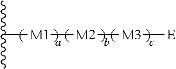

wherein M1 comprises a repeat unit comprising a hydrophilic sidechain;
wherein M2 comprises a repeat unit comprises a cholesterol-containing sidechain;
wherein M3 comprises a repeat unit comprising a hydrophilic sidechain;
a is an integer from 0 to 500;
b is an integer from 2 to 500;
c is an integer from 0 to 500;
E is absent, or represents a terminal unit optionally selected from the group consisting of dithioester, trithiocarbonate, xanthate;
L⁶ is absent, or represents a linking group; and
CM1 represents a first click motif.

In some aspects, CM1 can comprise an azide.

In some aspects, the modified hyaluronic acid can be covalently crosslinked by reaction of the copolymer defined by Formula IVA with a crosslinker defined by the structure below

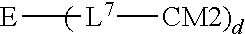

wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

In some aspects, the click reaction can comprise a strain-promoted alkyne-azide cycloaddition (SPAAC). In some aspects, CM2 can comprise an alkyne. In other aspects, the CM2 can comprise a dibenzocyclooctyne (DBCO) moiety.

In some aspects, E can comprise an oligomer or polymer.

Also provided herein are particles (e.g., nanoparticles and microparticles) formed from the modified hyaluronic acid polymers described herein. In some aspects, the particles can further comprise an active agent encapsulated within the particles. Also provided herein are hydrogels comprising the modified hyaluronic acid polymers described herein. In some aspects, the hydrogel can further comprise an active agent dispersed within the hydrogel. In some aspects, the hydrogel can further comprise one or more cells disposed on or within the hydrogel.

In some aspects, the active agent can comprise a small molecule therapeutic agent, a cosmetic agent, a diagnostic agent (e.g., detectable label), a native or synthetic polymer, a protein, a polypeptide, an oligonucleotide, antimicrobial particles (e.g., metal particles such as silver nanoparticles), minerals, a bioceramic, and/or a cell.

Particles and hydrogels that comprise active agent (e.g., pharmaceuticals or other bioactive moieties) are particularly useful in drug delivery applications, for example, as depots for sustained release, controlled release, or slow release of active agents.

The particles and hydrogels described herein can also be used in a variety of other applications, including as a scaffolding material for tissue engineering, for wound or fracture healing (either by itself, or as a substrate for cell delivery, e.g. the delivery of chondrocytes for repairing cartilage damage, or as a vehicle for delivering antimicrobial agents for preventing infections or treating infected wounds), joint damage, and cosmetic applications.

### DESCRIPTION OF DRAWINGS

FIG. 1 shows the ¹H NMR spectra of synthesized TBA HA azide in D₂O (trace a), Chol DBCO in CDCl₃ (trace b), Chol HA with DS of 6.4% (trace c) and DS of 1.0% (trace d) in a mixture of D₂O/DMSO-d6=1:2 (v/v).

FIG. 2 is a plot of the Degree of substitution (DS) and reaction efficiency for Chol HA as a function of the ratio of Chol DBCO to azide groups (D/A: 0.1 to 1).

FIG. 3 is a plot showing the FTIR spectra of HA azide and Chol HA with DS of 6.4%. Upon coupling with Chol DBCO, the azide peak at 2110 cm⁻¹ significantly reduced as expected.

FIG. 4 is a plot showing the shear viscosity at shear rate of 10 s⁻¹ for 1% of Chol HA in PBS (pH 7.4; n=3) as a function of D/A and DS. Inset: Pictures of respective Chol HA (1%) in PBS. With small amount of cholesterol introduced (2.5-3.8%), viscosity significantly increased due to the physical crosslinking of cholesterol, resulting in gelling in the absence of any chemical crosslinking.

FIGS. 5A-5B are plots showing the (FIG. 5A) viscosity and (FIG. 5B) thixotropy of Chol HA with different D/A ratios. Thixotropy of Chol HA with D/A of 0.25, 0.5 and 0.75 show hysteresis loops in their shear stress vs. shear rate plots.

FIGS. 6A-6B are plots showing the shear moduli of HA azide (FIG. 6A) and Chol HA (D/A=0.25) (FIG. 6B) monitored right after being mixed with 4-arm PEG-20k-DBCO (chemical crosslinker). HA azide (1 and 2%) with Mw of 50 and 100 kD were evaluated. Only the 2% of HA azide formulation formed a strong and free-standing gel with 4-arm PEG-20k-DBCO. The 100-kD HA azide gelled faster with 4-arm PEG-20k-DBCO (gelling time of 5-6 min) than the 50-kD HA azide (gelling time 9-10 min). After incorporation of cholesterol to HA azide, 1.5% of Chol HA (D/A 0.25) was able to form a free-standing gel after chemical crosslinking with 4-arm PEG-20k-DBCO but with a longer gelling time (˜20 min).

FIG. 7A is a CLSM image of Chol HA nanoparticles (NPs) with D/A=1 in PBS, incorporated with 0.4% (w/w) of Nile red.

FIG. 7B is a plot of the DLS of Chol HA (D/A=1) before and after incubation with hyaluronidase (HYAL) (7U, 37° C. for 4 hr; n=3), showing that the stable Chol HA NPs were resistant to enzyme digestion.

FIG. 8A is a photograph of Chol HA (0.25%) with D/A from 0.1 to 1 (left to right) in PBS (pH 7.4) after incorporation of Nile red (0.4%, w/w, relative to Chol HA.

FIG. 8B shows the UV-Vis spectra of the solutions photographed in FIG. 8A.

FIG. 8C is a plot showing the absorbance at 560 nm of the aqueous Chol HA with D/A=1, incorporated with Nile red from 0.08 to 1.2% (w/w, n=3), revealing a loading capacity of Nile red ˜0.4% (w/w).

FIG. 9 is a plot showing the UV-vis spectrum of Chol HA NPs (D/A=1; NPs-filtered), Chol HA NPs/vitamin C mixture (NPs-VitC-filtered) and free vitamin C (Free VitC) recorded after overnight incubation in the dark at RT and filtration through a membrane (pore size: 0.1 μm). The peak at 265 nm, characteristic for vitamin C, was only detected when it was stabilized by Chol HA NPs after overnight incubation.

FIGS. 10A-10B show fluorescence (FIG. 10A) and bright field optical (FIG. 10B) micrographs of rat bone marrow stromal cells (MSCs) after 4-hr incubation with Nile red (NR)-loaded Chol HA NPs (250 μg/mL).

FIG. 11 depicts the non-covalent interactions of Chol HA with 0% (A), 2.5-3.8% (B) and 6.4% (C) of cholesterol units in PBS. With the increase of cholesterol units, viscosity of Chol HA first increased due to the interchain hydrophobic (Chol-Chol) interactions. Above 3.8% of cholesterol, viscosity dropped due to the formation of nanoparticles (NPs), driven by intrachain hydrophobic Chol-Chol clustering.

FIG. 12A schematically illustrates the chemical structure of an example cholesteryl-modified HA made with isolated cholesterol and HA azide. The line between the 2 SPAAC adducts can represent any oligomer or polymer carrying at least 2 DBCO end groups. While illustrated as a bivalent crosslinker in FIG. 12A, the oligomer or polymer can be di-, tri-, tetra- or star-branched, incorporating varying numbers of DBCO moieties.

FIG. 12B illustrates different interactions present in type I cholesterol-modified HA hydrogels with high and low DS of cholesterol.

FIG. 13A schematically illustrates the chemical structure of cholesteryl-modified HA made with cholesterol oligomers and HA azide. The hydrophilic side chain copolymerized to alternate with the cholesterol side chain in the oligomer can be of any length or structure. In some cases, the sidechain can comprise an oligomer bearing a combination of short hydrophilic side chain and cholesterol side chains in varying ratios, such as 1:10 to 10:1. The RAFT copolymerization can also be carried out with the corresponding (meth)acrylates or (meth)acrylamides, even though the copoymerization of methacrylates is shown in FIG. 13A.

FIG. 13B illustrates the different interactions present in type II cholesterol HA hydrogels with high and low DS of cholesterol.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

“Active Agent”, as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

“Effective amount” or “therapeutically effective amount”, as used herein, refers to an amount of polymer-drug conjugate effective to alleviate, delay onset of, or prevent one or more symptoms of a disease or disorder being treated by the active agent, and/or an amount of polymer-drug conjugate effective to produce a desired diagnostic signal.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

“Biodegradable Polymer” as used herein, generally refers to a polymer that will degrade or erode by enzymatic action or hydrolysis under physiologic conditions to smaller units or chemical species that are capable of being metabolized, eliminated, or excreted by the subject. The degradation time is a function of polymer composition, morphology, such as porosity, particle dimensions, and environment.

“Nanoparticle”, as used herein, generally refers to a particle having a diameter, such as an average diameter, from about 10 nm up to but not including about 1 micron, preferably from 100 nm to about 1 micron. The particles can have any shape. Nanoparticles having a spherical shape are generally referred to as “nanospheres”.

“Microparticle”, as used herein, generally refers to a particle having a diameter, such as an average diameter, from about 1 micron to about 100 microns, preferably from about 1 to about 50 microns, more preferably from about 1 to about 30 microns, most preferably from about 1 micron to about 10 microns. The microparticles can have any shape. Microparticles having a spherical shape are generally referred to as “microspheres”.

“Molecular weight” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

“Pharmaceutically Acceptable”, as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “hydrogel” refers to a water-containing three-dimensional hydrophilic polymer network or gel in which the water is the continuous phase and in which the water content is greater than 50% (w/w).

The term “hyaluronic acid polymer” refers to a polymer comprising repeat disaccharide subunits of hyaluronan, where the repeat units may be derivatized at one or more positions of the D-glucuronic acid and/or the D-N-acetylglucosamine unit of the disaccharide repeat subunit A hyaluronic acid polymer is meant to encompass hyaluronic acid (also referred to as hyaluronan), derivatized hyaluronic acid, salts forms, hyaluronic acid linker complexes, and hyaluronic acid conjugates. The terms “hyaluronic acid derivative” or “derivatized hyaluronic acid” or “modified hyaluronic acid” refers to a hyaluronic acid polymer which has been derivatized by reaction with, e g, one or more chemical moieties.

Ranges of values defined herein include all values within the range as well as all sub-ranges within the range. For example, if the range is defined as an integer from 0 to 10, the range encompasses all integers within the range and any and all subranges within the range, e.g., 1-10, 1-6, 2-8, 3-7, 3-9, etc.

### Modified Hyaluronic Acids

Described herein are covalently-modified hyaluronic acid polymers. The covalently-modified hyaluronic acid polymers can be functionalized, for example via click chemistry, to include one or more cholesterol moieties. Optionally, the covalently-modified hyaluronic acid polymers can be covalently crosslinked (e.g., using a bifunctional or polyfunctional crosslinker which covalently crosslinks the covalently-modified hyaluronic acid polymers via click reactions). These modified hyaluronic acid polymers can prepare particles (e.g., nanoparticles and/or microparticles) and 3D viscoelastic hydrogels.

In some examples, cholesterol-modified HA can be used to prepare 3D viscoelastic hydrogels with or without the combination of covalent crosslinking (e.g., using click chemistry such as strain-promoted alkyne-azide cycloaddition (SPAAC)). The cholesterol moieties can enable physical (reversible) cross-linking and facilitate interactions with encapsulated cells and enable the retention and release of specific biotherapeutics. Cholesterol, a key component of lipid rafts within cell membranes, is known for excellent biocompatibility and cell permeability. It has been introduced onto hydrophilic polymers for hydrophobic drug delivery where the hydrophobic interaction of cholesterol moieties leads to the self-assembly of polymers in aqueous media, usually in the form of nanoparticles.

In the modified HA polymers described herein, the cholesterol moiety can be introduced in a stoichiometrically controlled manner through well-defined oligomer brushes and/or isolated units, enabling a broad range of tuning of viscoelasticity and physical/biological modulations of the 3D synthetic cellular niches environment. The ratio of physical to covalent cross-linking can thus be prospectively and stoichiometrically controlled.

The cholesteryl HA hydrogels described herein can exhibit cholesterol-cholesterol physical cross-linking within the 3D amphiphilic hydrated network in the form of hydrophobic micropockets, where their microstructures and affinity of association (strength of physical crosslinking) are all tunable depending on the introduced form and content of the synthetic cholesteryl modalities. This is in contrast to other crosslinked HA hydrogels, which utilize chemical crosslinking or physical entanglement of polymer chains to modulate the 3D hydrogel environment.

Given the excellent biocompatibility of HA and cholesterol, plus the physical cross-linking from cholesterol hydrophobic interaction, the resulting hydrogels can be beneficial to 3D cell culture and in vivo tissue regeneration. Cholesterol, in either isolated or oligomer form, can be coupled to with HA via a covalent linkage (e.g., using click chemistry such as SPAAC).

In some embodiments, the cholesterol moieties can be attached to HA backbone with a controlled low degree of substitution (DS) to favor physical crosslinking within 3D hydrogel rather than facilitating the formation of self-assembled particulates.

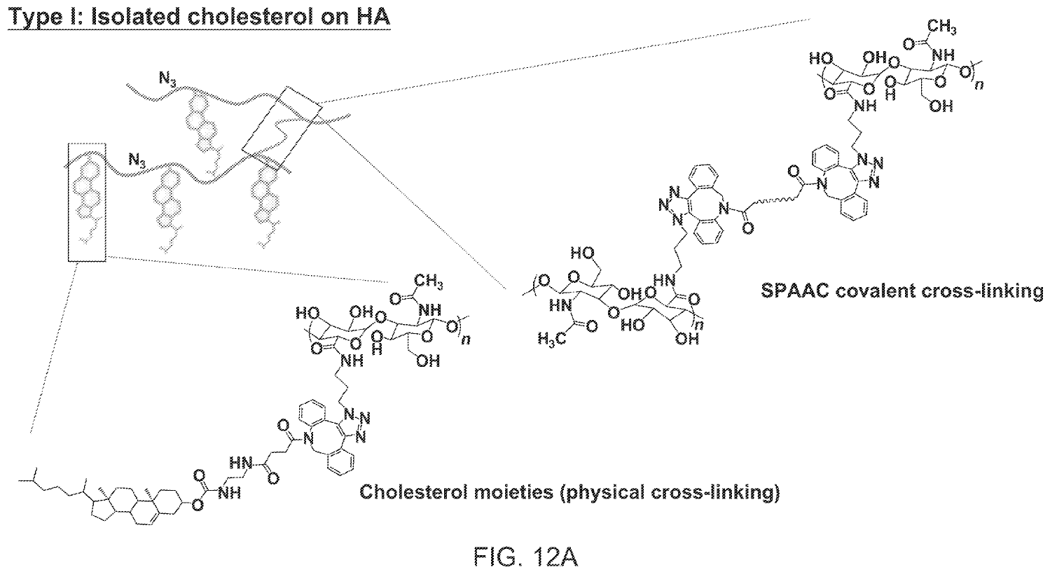

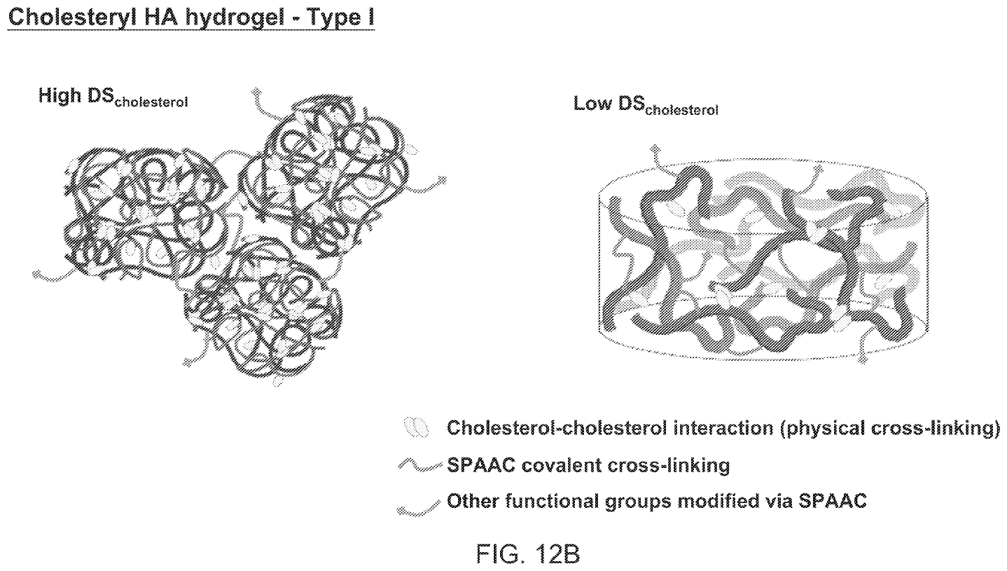

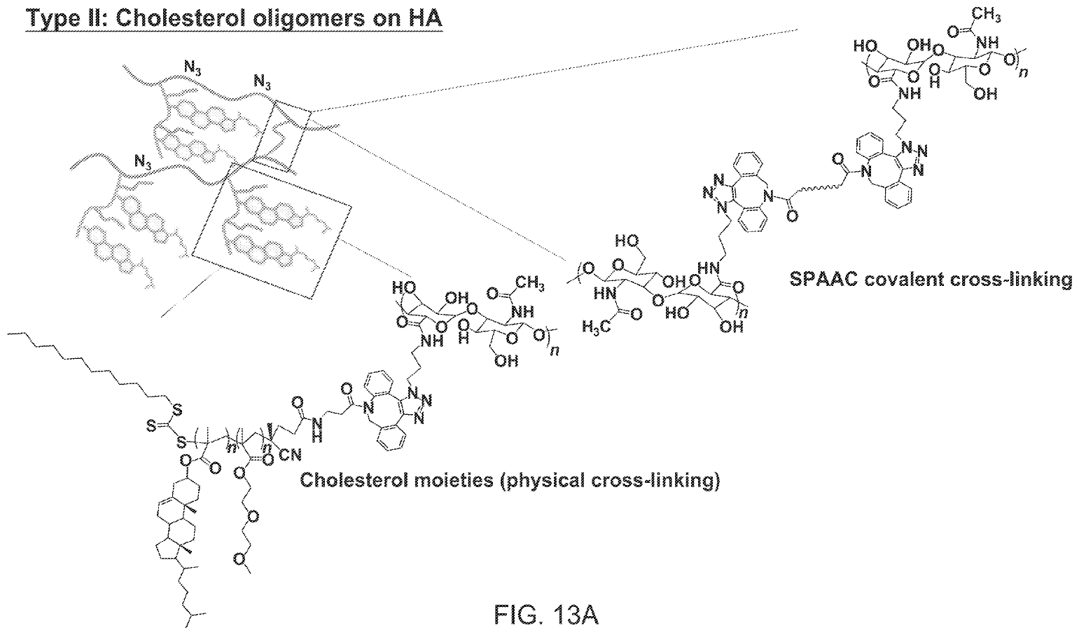

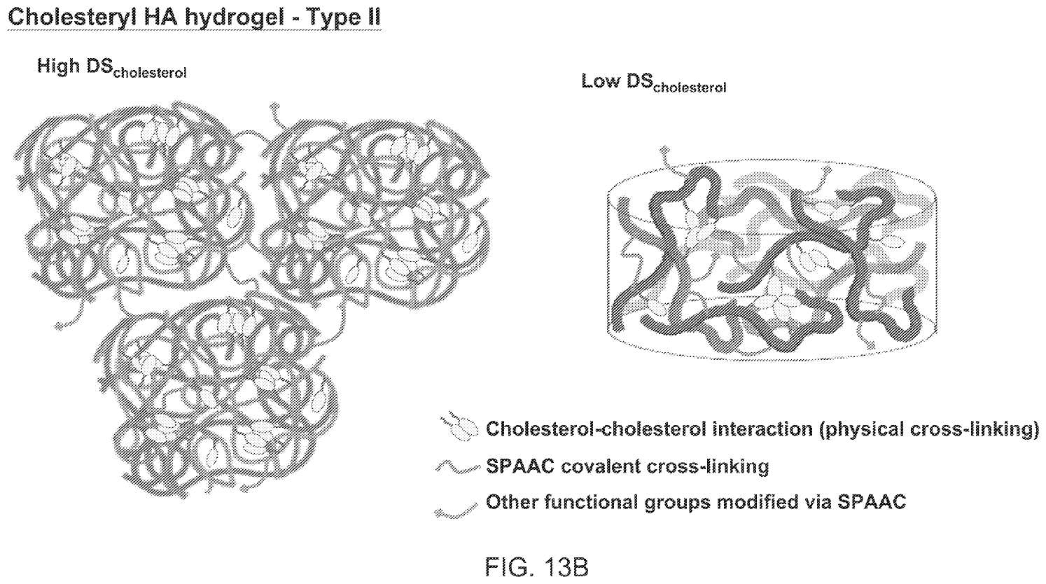

For example, cholesterol-modified HA can be prepared using two types of cholesterol ligands. By way of exemplification, isolated cholesterol (see FIG. 12A) or cholesterol oligomers (degree of polymerization/DP: 2-50) (see FIG. 13A) can be used to introduce cholesterol moieties. Optionally, in order to improve the water solubility and flexibility, hydrophilic chains such as di(ethylene glycol)methyl ether methacrylate can be copolymerized in the cholesterol oligomers (FIG. 13A).

The coupling of cholesterol ligands onto HA can be carried out via click chemistry (e.g., SPAAC). The DS of cholesterol on HA can be kept low, for example, ranging from 0.01 to 0.1. In some embodiments, the molecular weight of the backbone HA can be from 50 to 1000 kD. In some embodiments, the cholesterol can be covalently crosslinked, for example, using click chemistry. In certain embodiments, SPAAC covalent cross-linking can be used. The crosslink density can be varied to tune the physical properties of the HA. For example, in some embodiments, SPAAC covalent cross-linking can be introduced with different SPAAC/cholesterol-cholesterol crosslinking ratios ranging from 0 to 99.9%. Additionally, if desired, click motifs introduced onto the HA backbone can be readily derivatized using click chemistry (e.g., to modify the hydrophobicity/hydrophilicity of the modified HA). By way of example, azide groups introduced onto the HA backbone can be further modified with other functional groups of interest via SPAAC. In some embodiments, an active agent (e.g., a small molecule therapeutic agent, a nucleotide, a protein, or a carbohydrate) can be covalently attached to the HA via click chemistry.

The cholesterol HA hydrogels with isolated cholesterol are depicted in FIG. 12B, where hydrogels with higher DS of cholesterol are more likely to include packed cholesterol clusters in the network while hydrogels with lower DS are more likely to adopt a less heterogeneous structure. As for cholesterol HA hydrogels bearing cholesterol oligomers, the hydrophobic liquid crystalline-like domains are strengthened by aligned/interdigitated cholesterol units on the oligomer pendants (FIG. 13B). In these amphiphilic hydrogels, the dynamic nature of the hydrophobic domains can provide a mechanism for stress relaxation (viscoelasticity), and allow for adhesion by encapsulated cells, promote certain cellular interactions, and attract exogenous biotherapeutics or enrich endogenously secreted factors.

Cholesteryl-modified HA can be in the form of viscous liquid (sol) upon dispersing in aqueous solution. In this stage, factors such as the type of cholesterol ligands (isolated or oligomer), DS, and sonication can dictate the morphology, size, and distribution of the cholesterol moieties in cholesteryl HA. When present, unreacted click motifs introduced along the backbone of the HA (e.g., unreacted azide groups) can provide a synthetic handle to allow for subsequent covalent cross-linking (e.g., via click chemistry such as SPAAC). This crosslinking can induce hydrogel formation (a sol-gel transition). By way of example, a polyvalent crosslinker (e.g., 4-arm PEG20k-amide-DBCO) was synthesized and used as a crosslinker for SPAAC covalent cross-linking.

The resulting cholesteryl-modified HA properties were used to prepare particles (e.g., nanoparticles and/or microparticles) and 3D viscoelastic hydrogels, as depicted in FIG. 11 and discussed in more detail and exemplified below.

In some embodiments, provided herein are modified hyaluronic acid polymers comprising one or more covalently modified monomers defined by Formula I below

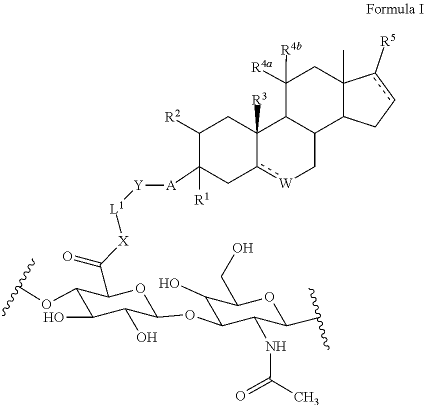

wherein
[symbol] denotes carbon-carbon bond or carbon-carbon double bond;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is NH, O, or S;
R^{a} is

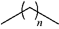

wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is

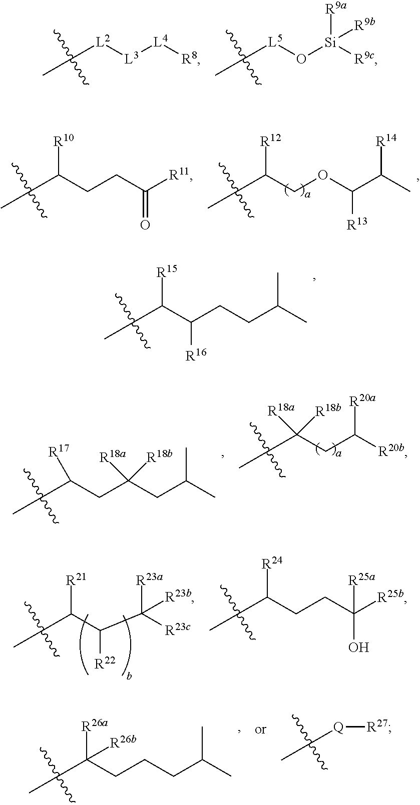

L² is absent,

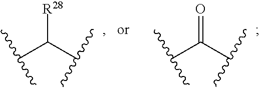

R²⁸ is absent or C₁-C₆ alkyl;
L³ is absent, or

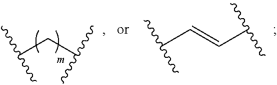

m is an integer 1, 2, or 3;
L⁴ is absent,

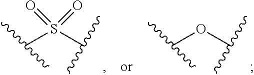

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or

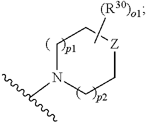

R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form

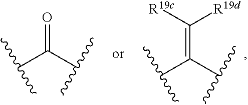

were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

In some embodiments of Formula I, R⁵ can be

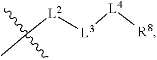

wherein
L² is absent,

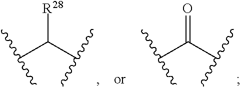

R²⁸ is absent or C₁-C₆ alkyl;
L³ is absent, or

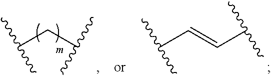

m is an integer 1, 2, or 3;
L⁴ is absent,

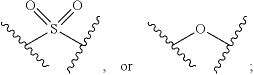

and
R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl.

In some embodiments of Formula I, R¹ is C₁-C₆ alkyl. In certain embodiments of Formula I, R¹ is —CH₃.

In some embodiments of Formula I, A is O.

In some embodiments of Formula I, L² is

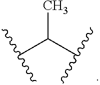 [SMILES: *C(*)C]

In some embodiments of Formula I, L³ is

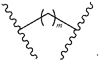

In some embodiments of Formula I, m is 1. In some embodiments of Formula I, m is 2. In some embodiments of Formula I, m is 3.

In some embodiments of Formula I, L⁴ is absent. In other embodiments of Formula I, L⁴ is

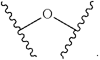 [SMILES: *O*]

In some embodiments of Formula I, R⁸ is methyl, ethyl, n-propyl, isopropyl, n-butyl, isobutyl, t-butyl, pentyl, hexyl, heptyl, octyl, nonyl, or decyl. In certain embodiments of Formula I, R⁸ is isopropyl.

In some embodiments of Formula I, X is NH.

In some embodiments of Formula I, Y is —NR^{a}C(═O)—.

In some embodiments of Formula I, L¹ comprises a moiety formed by a strain-promoted alkyne-azide cycloaddition (SPAAC). In certain embodiments of Formula I, L¹ comprises a moiety formed by reaction (e.g., a SPAAC) of an azide moiety with a dibenzocyclooctyne (DBCO) moiety.

In some embodiments of Formula I, L¹ further comprises an oligomer or polymer (e.g., an oligoalkylene oxide or polyalkylene oxide).

In some embodiments, the modified hyaluronic acid can comprise a random copolymer defined by Formula II below

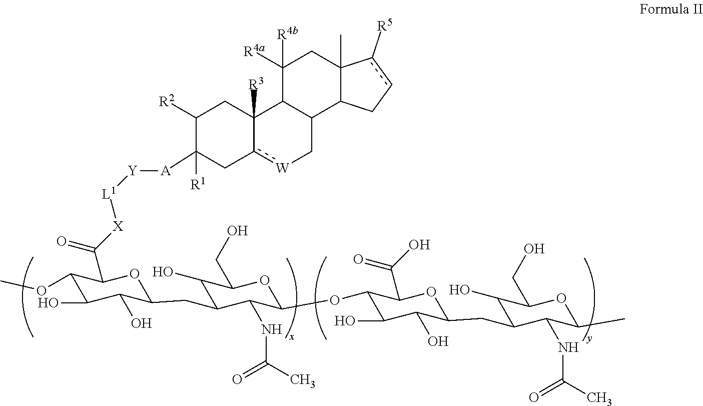

wherein
x and y are each independently integers from 1 to 2500, wherein x+y is no more than 2500, and wherein x and y represent the relative portion of each monomer within the random copolymer;
[symbol] denotes carbon-carbon bond or carbon-carbon double bond;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is NH, O, or S;
R^{a} is

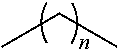

wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is

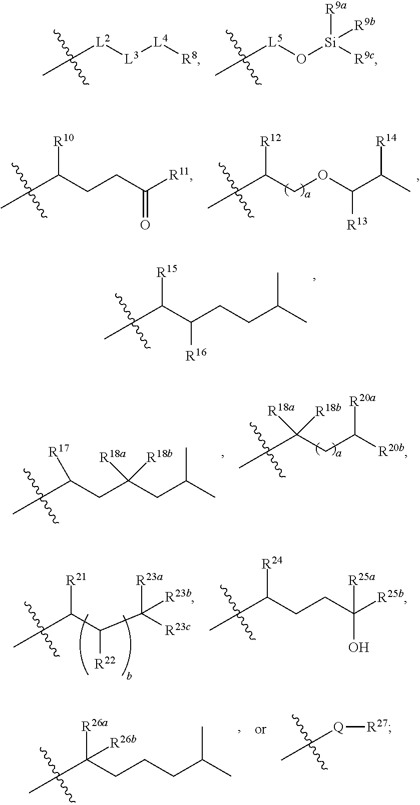

L² is absent,

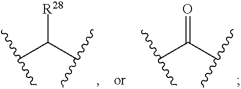

R²⁸ is absent or C₁-C₆ alkyl;
L³ is absent, or

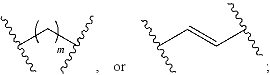

m is an integer 1, 2, or 3;
L⁴ is absent,

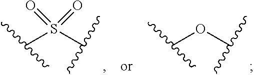

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or

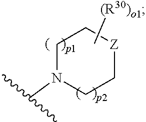

R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form

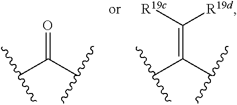

were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

In some embodiments of Formula II, R⁵ can be

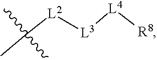

wherein
L² is absent,

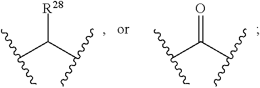

R²⁸ is absent or C₁-C₆ alkyl;
L³ is absent, or

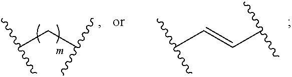

m is an integer 1, 2, or 3;
L⁴ is absent,

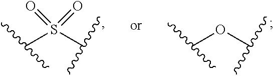

and
R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl.

In some embodiments of Formula II, R¹ is C₁-C₆ alkyl. In certain embodiments of Formula II, R¹ is —CH₃.

In some embodiments of Formula II, A is O.

In some embodiments of Formula II, L² is

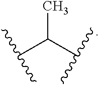 [SMILES: *C(*)C]

In some embodiments of Formula II, L³ is

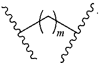

In some embodiments of Formula II, m is 1. In some embodiments of Formula II, m is 2. In some embodiments of Formula II, m is 3.

In some embodiments of Formula II, L⁴ is absent. In other embodiments of Formula II, L⁴ is

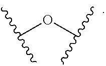 [SMILES: *O*]

In some embodiments of Formula II, R⁸ is methyl, ethyl, n-propyl, isopropyl, n-butyl, isobutyl, t-butyl, pentyl, hexyl, heptyl, octyl, nonyl, or decyl. In certain embodiments of Formula II, R⁸ is isopropyl.

In some embodiments of Formula II, X is NH.

In some embodiments of Formula II, Y is —NR^{a}C(═O)—.

In some embodiments of Formula II, L¹ comprises a moiety formed by a strain-promoted alkyne-azide cycloaddition (SPAAC). In certain embodiments of Formula II, L¹ comprises a moiety formed by reaction (e.g., a SPAAC) of an azide moiety with a dibenzocyclooctyne (DBCO) moiety.

In some embodiments of Formula II, L¹ further comprises an oligomer or polymer (e.g., an oligoalkylene oxide or polyalkylene oxide).

In some embodiments of Formula II, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula II, x can be from 1 to 2400 (e.g., from 1 to 1500, from 1 to 1000, from 25 to 1000, or from 25 to 500).

In some embodiments of Formula II, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, y can be 2400 or less (e.g., 2300 or less, 2200 or less, 2100 or less, 2000 or less, 1900 or less, 180 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula II, y can be from 10 to 2400 (e.g., from 10 to 1500, from 10 to 1000, from 25 to 1000, or from 25 to 500).

In some embodiments of Formula II, x+y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, x+y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x+y can range from any of the minimum values described above to any of the maximum values described above. For example, in some embodiments of Formula II, x+y can be from 10 to 2400 (e.g., from 10 to 1500, from 10 to 1000, from 25 to 1000, or from 25 to 500).

In some embodiments, the modified hyaluronic acid can be covalently crosslinked. In certain embodiments, the modified hyaluronic acid can be covalently crosslinked using click chemistry.

In some embodiments, the modified hyaluronic acid can comprise a random copolymer defined by Formula III below

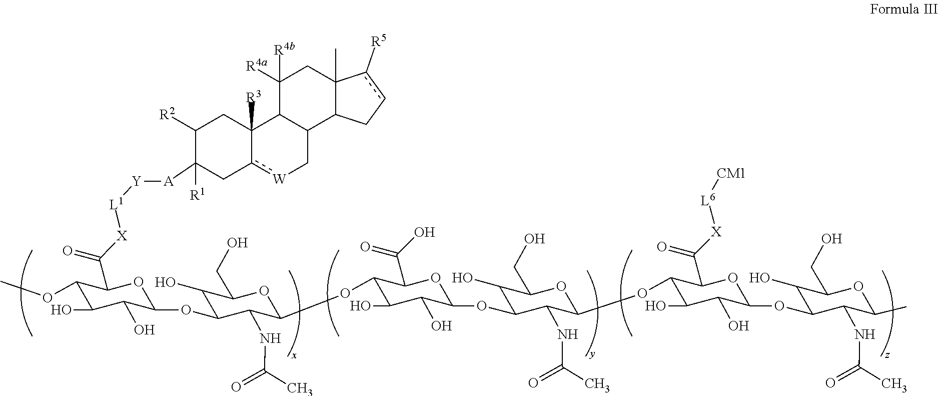

wherein
x, y, and z are each independently integers from 1 to 2500, wherein x, y, and z represent the relative portion of each monomer within the random copolymer, and wherein x+y+z is no more than 2500;
[symbol] denotes carbon-carbon bond or carbon-carbon double bond;
L⁶ is absent, or represents a linking group;
CM1 represents a first click motif;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is, independently for each occurrence, NH, O, or S;
R^{a} is

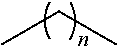

wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR⁴ or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is

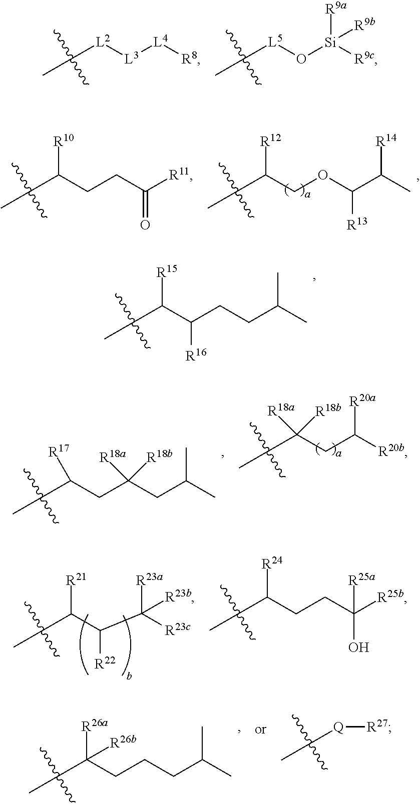

L² is absent,

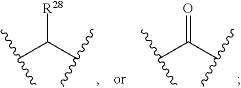

R²⁸ is absent or C₁-C₆ alkyl;

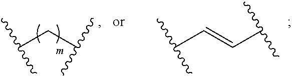

m is an integer 1, 2, or 3;
L⁴ is absent,

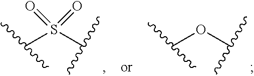

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or

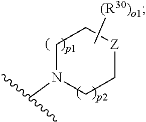

R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form

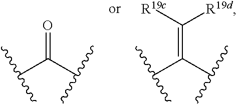

were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

In some embodiments, CM1 can comprise an azide.

In some embodiments, the modified hyaluronic acid can be covalently crosslinked by reaction of the copolymer defined by Formula III with a crosslinker defined by the structure below

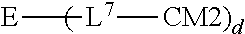

wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

In some embodiments, the click reaction can comprise a strain-promoted alkyne-azide cycloaddition (SPAAC). In some embodiments, CM2 can comprise an alkyne. In other aspects, the CM2 can comprise a dibenzocyclooctyne (DBCO) moiety.

In some aspects, E can comprise an oligomer or polymer.

In some embodiments of Formula III, R⁵ can be

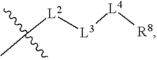

wherein
L² is absent,

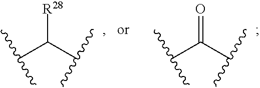

R²⁸ is absent or C₁-C₆ alkyl,
L³ is absent, or

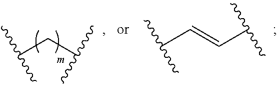

m is an integer 1, 2, or 3;
L⁴ is absent,

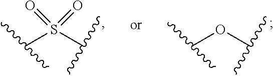

and
R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl.

In some embodiments of Formula III, R¹ is C₁-C₆ alkyl. In certain embodiments of Formula III, R¹ is —CH₃.

In some embodiments of Formula III, A is O.

In some embodiments of Formula III, L² is

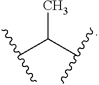 [SMILES: *C(*)C]

In some embodiments of Formula III, L³ is

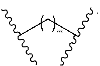

In some embodiments of Formula III, m is 1. In some embodiments of Formula III, m is 2. In some embodiments of Formula III, m is 3.

In some embodiments of Formula III, L⁴ is absent. In other embodiments of Formula III, L⁴ is

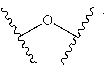 [SMILES: *O*]

In some embodiments of Formula III, R⁸ is methyl, ethyl, n-propyl, isopropyl, n-butyl, isobutyl, t-butyl, pentyl, hexyl, heptyl, octyl, nonyl, or decyl. In certain embodiments of Formula III, R⁸ is isopropyl.

In some embodiments of Formula III, X is NH.

In some embodiments of Formula III, Y is —NR^{a}C(═O)—.

In some embodiments of Formula III, L¹ comprises a moiety formed by a strain-promoted alkyne-azide cycloaddition (SPAAC). In certain embodiments of Formula III, L¹ comprises a moiety formed by reaction (e.g., a SPAAC) of an azide moiety with a dibenzocyclooctyne (DBCO) moiety.

In some embodiments of Formula III, L¹ further comprises an oligomer or polymer (e.g., an oligoalkylene oxide or polyalkylene oxide).

In some embodiments of Formula III, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula III, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula III, x can be from 1 to 2400 (e.g., from 1 to 1500, from 1 to 1000, from 25 to 1000, or from 25 to 500).

In some embodiments of Formula III, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula III, y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula III, y can be from 10 to 2400 (e.g., from 10 to 1500, from 10 to 1000, from 25 to 1000, or from 25 to 500).

In some embodiments of Formula III, z can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula III, z can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

z can range from any of the minimum values described above to any of the maximum values described above. For example, in some embodiments of Formula III, z can be from 1 to 2400 (e.g., from 1 to 1500, from 1 to 1000, from 25 to 1000, or from 25 to 500).

In some embodiments of Formula III, x+y+z can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula III, x+y+z can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, or less, or 10 or less).

x+y+z can range from any of the minimum values described above to any of the maximum values described above. For example, in some embodiments of Formula III, x+y+z can be from 10 to 2400 (e.g., from 10 to 1500, from 10 to 1000, from 25 to 1000, or from 25 to 500).

In some embodiments, the modified hyaluronic acid can comprise one or more covalently modified monomers defined by Formula IA

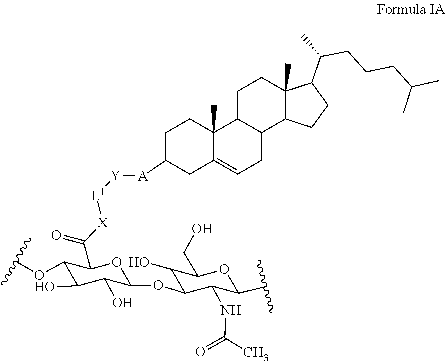

wherein
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two click motifs;
X is NH, O, or S; and
R^{a} is

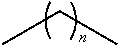

wherein n is an integer from 0 to 12.

In some embodiments of Formula IA, X is NH.

In some embodiments of Formula IA, Y is —NR^{a}C(═O)—.

In some embodiments of Formula IA, L¹ comprises a moiety formed by a strain-promoted alkyne-azide cycloaddition (SPAAC). In certain embodiments of Formula IA, L¹ comprises a moiety formed by reaction (e.g., a SPAAC) of an azide moiety with a dibenzocyclooctyne (DBCO) moiety.

In some embodiments of Formula IA, L¹ further comprises an oligomer or polymer (e.g., an oligoalkylene oxide or polyalkylene oxide).

In some embodiments, the modified hyaluronic acid can comprise a random copolymer defined by Formula IIA below

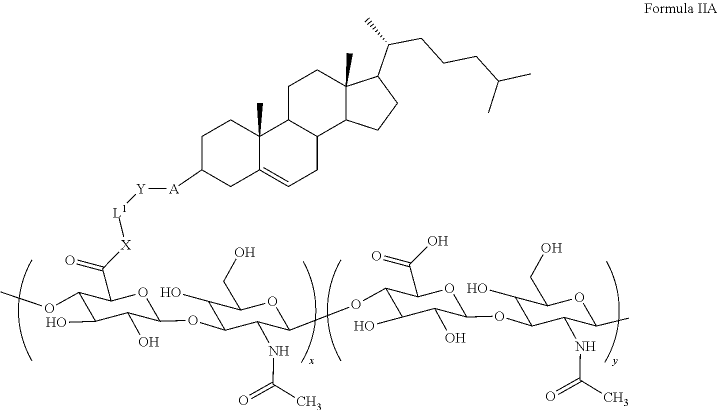

wherein
x and y are each independently integers from 1 to 2500, wherein x and y represent the relative portion of each monomer within the random copolymer, and wherein x+y+z is no greater than 2500;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is NH, O, or S; and
R^{a} is

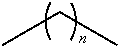

wherein n is an integer from 0 to 12.

In some embodiments of Formula IIA, X is NH.

In some embodiments of Formula IIA, Y is —NR^{a}C(═O)—.

In some embodiments of Formula IIA, L¹ comprises a moiety formed by a strain-promoted alkyne-azide cycloaddition (SPAAC). In certain embodiments of Formula IIA, L¹ comprises a moiety formed by reaction (e.g., a SPAAC) of an azide moiety with a dibenzocyclooctyne (DBCO) moiety.

In some embodiments of Formula IIA, L¹ further comprises an oligomer or polymer (e.g., an oligoalkylene oxide or polyalkylene oxide).

In some embodiments of Formula IA, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IIA, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula IIA, x can be from 1 to 2400 (e.g., from 1 to 1500, from 1 to 1000, from 25 to 1000, or from 25 to 500).

In some embodiments of Formula IA, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IIA, y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula IIA, y can be from 10 to 2400 (e.g., from 10 to 1500, from 10 to 1000, from 25 to 1000, or from 25 to 500).

In some embodiments of Formula IA, x+y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IIA, x+y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x+y can range from any of the minimum values described above to any of the maximum values described above. For example, in some embodiments of Formula IIA, x+y can be from 10 to 2400 (e.g., from 10 to 1500, from 10 to 1000, from 25 to 1000, or from 25 to 500).

In some embodiments, the modified hyaluronic acid can comprise a random copolymer defined by Formula IIIA below

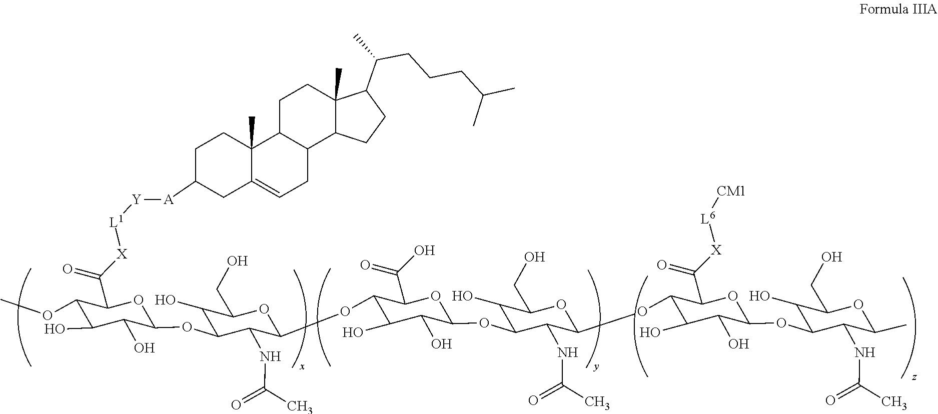

wherein
x, y, and z are each independently integers from 1 to 2500, wherein x, y, and z represent the relative portion of each monomer within the random copolymer, and wherein x+y+z is no greater than 2500;
L⁶ is absent, or represents a linking group;
CM1 represents a first click motif;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is, independently for each occurrence, NH, O, or S;
R^{a} is

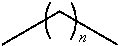

wherein n is an integer from 0 to 12.

In some embodiments, CM1 can comprise an azide.

In some embodiments, the modified hyaluronic acid can be covalently crosslinked by reaction of the copolymer defined by Formula IIIA with a crosslinker defined by the structure below

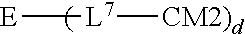

wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

In some embodiments of Formula IIIA, X is NH.

In some embodiments of Formula IIIA, Y is —NR^{a}C(═O)—.

In some embodiments of Formula IIIA, L¹ comprises a moiety formed by a strain-promoted alkyne-azide cycloaddition (SPAAC). In certain embodiments of Formula IIIA, L¹ comprises a moiety formed by reaction (e.g., a SPAAC) of an azide moiety with a dibenzocyclooctyne (DBCO) moiety.

In some embodiments of Formula IIIA, L further comprises an oligomer or polymer (e.g., an oligoalkylene oxide or polyalkylene oxide).

In some embodiments of Formula IIIA, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IIIA, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula IIIA, x can be from 1 to 2400 (e.g., from 1 to 1500, from 1 to 1000, from 25 to 1000, or from 25 to 500).

In some embodiments of Formula IIIA, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IIIA, y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula IIIA, y can be from 10 to 2400 (e.g., from 10 to 1500, from 10 to 1000, from 25 to 1000, or from 25 to 500).

In some embodiments of Formula IIIA, z can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IIIA, z can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

z can range from any of the minimum values described above to any of the maximum values described above. For example, in some embodiments of Formula IIIA, z cm be from 1 to 2400 (e.g., from 1 to 1500, from 1 to 1000, from 25 to 1000, or from 25 to 500).

In some embodiments of Formula IIIA, x+y+z can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IIIA, x+y+z can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, or less, 15 or less, or 10 or less).

x+y+z can range from my of the minimum values described above to any of the maximum values described above. For example, in some embodiments of Formula IIIA, x+y+z can be from 10 to 2400 (e.g., from 10 to 1500, from 10 to 1000, from 25 to 1000, or from 25 to 500).

Also provided are modified hyaluronic acid polymers that comprise one or more covalently modified monomers defined by Formula IV

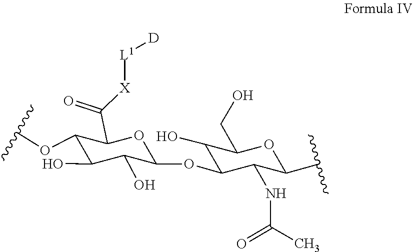

wherein
L¹ is a linking group comprising a moiety formed by the chemical reaction of two click motifs;
X is, independently for each occurrence, NH, O, or S;
D represents an oligomer or copolymer (block copolymer or random copolymer) defined by the formula below

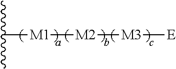

wherein M1 comprises a repeat unit comprising a hydrophilic sidechain;
wherein M2 comprises a repeat unit comprises a cholesterol-containing sidechain;
wherein M3 comprises a repeat unit comprising a hydrophilic sidechain
a is an integer from 0 to 500;
b is an integer from 2 to 500;
c is an integer from 0 to 500; and
E is absent, or represents a terminal unit optionally selected from the group consisting of dithioester, trithiocarbonate, xanthate.

In some embodiments, M1, M2, and M3 are independently repeat units obtainable by polymerization of a (meth)acrylate or (meth)acrylamide monomer, such as reversible addition-fragmentation chain-transfer polymerization (RAFT) of a (meth)acrylate or (meth)acrylamide monomer.

In some embodiments, a can be 0. In other embodiments, a can be at least 1 (e.g., at least 5, at least 10, at least 25, at least 50, at least 75, at least 100, at least 150, at least 200, at least 250, at least 300, at least 350, at least 400, or at least 450). In some embodiments, a can be 500 or less (e.g., 450 or less, 400 or less, 350 or less, 300 or less, 250 or less, 200 or less, 150 or less, 100 or less, 75 or less, 50 or less, 25 or less, 10 or less, or 5 or less).

a can be from any of the minimum values described above to any of the maximum values described above. For example, in some embodiments, a can be from 0 to 500 (e.g., from 0 to 100, from 1 to 500, or from 1 to 100).

In other embodiments, b cm be at least 2 (e.g., at least 5, at least 10, at least 25, at least 50, at least 75, at least 100, at least 150, at least 200, at least 250, at least 300, at least 350, at least 400, or at least 450). In some embodiments, b can be 500 or less (e.g., 450 or less, 400 or less, 350 or less, 300 or less, 250 or less, 200 or less, 150 or less, 100 or less, 75 or less, 50 or less, 25 or less, 10 or less, or 5 or less).

b can be from any of the minimum values described above to any of the maximum values described above. For example, in some embodiments, b can be from 2 to 500 (e.g., from 2 to 100).

In some embodiments, c can be 0. In other embodiments, c can be at least 1 (e.g., at least 5, at least 10, at least 25, at least 50, at least 75, at least 100, at least 150, at least 200, at least 250, at least 300, at least 350, at least 400, or at least 450). In some embodiments, c can be 500 or less (e.g., 450 or less, 400 or less, 350 or less, 300 or less, 250 or less, 200 or less, 150 or less, 100 or less, 75 or less, 50 or less, 25 or less, 10 or less, or 5 or less).

c can be from any of the minimum values described above to any of the maximum values described above. For example, in some embodiments, c can be from 0 to 500 (e.g., from 0 to 100, from 1 to 500, or from 1 to 100).

In some embodiments, at least one of a and c is from 2 to 500 (e.g., from 2 to 100).

In some embodiments, M1 and M3 are independently repeat units obtainable by polymerization of a (meth)acrylate or (meth)acrylamide monomer comprising a hydrophilic oligomer or polymer sidechain (e.g., an oligo- or polyethylene glycol sidechain), such as reversible addition-fragmentation chain-transfer polymerization (RAFT) of a (meth)acrylate or (meth)acrylamide monomer comprising a hydrophilic oligomer or polymer sidechain, such as an oligo- or polyethylene glycol sidechain.

In some embodiments, M2 is a repeat unit obtainable by polymerization of a (meth)acrylate or (meth)acrylamide monomer comprising a (meth)acrylate or (meth)acrylamide monomer comprising a sidechain defined by the formula below, such as reversible addition-fragmentation chain-transfer polymerization (RAFT) of a (meth)acrylate or (meth)acrylamide monomer comprising a sidechain defined by the formula below

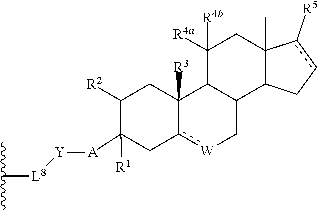

A is absent, or is O or S;
Y is absent, or represents —O(C═O)—, —C(═O)—, —S(O)ₓ—, —C(═O)R^{a}C(═O)—, —NR^{a}C(═O)—, —NC(═O)R^{a}C(═O)NR^{a}C(═O);
[symbol] denotes carbon-carbon bond or carbon-carbon double bond;
L⁸ is absent, or is a linking group;
R^{a} is

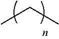

wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;

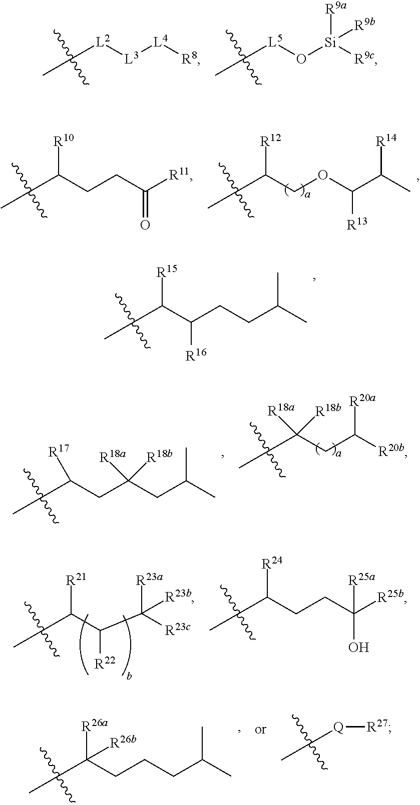

L² is absent,

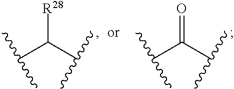

R² is absent or C₁-C₆ alkyl;
L³ is absent, or

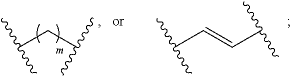

m is an integer 1, 2, or 3;
L⁴ is absent,

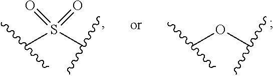

R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}OR^{29b}, or

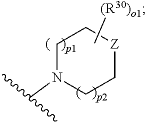

R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form

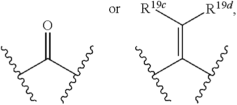

were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

In some embodiments, M2 is a repeat unit obtainable by polymerization of a (meth)acrylate or (meth)acrylamide monomer comprising a (meth)acrylate or (meth)acrylamide monomer comprising a sidechain defined by the formula below, such as reversible addition-fragmentation chain-transfer polymerization (RAFT) of a (meth)acrylate or (meth)acrylamide monomer comprising a sidechain defined by the formula below

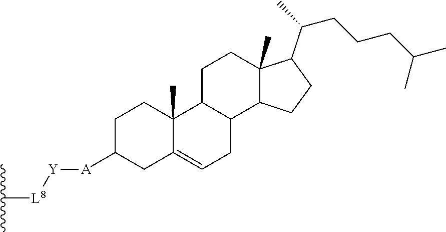

A is absent, or is O or S;
Y is absent, or represents —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; and
L⁸ is absent, or is a linking group.

In some embodiments, the modified hyaluronic acid can comprise a random copolymer defined by Formula IVA below

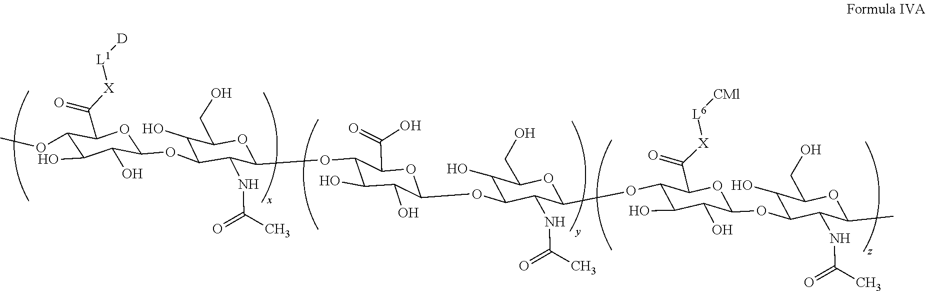

wherein
x, y, and z are each independently integers from 1 to 2500, wherein x, y, and z represent the relative portion of each monomer within the random copolymer, and wherein x+y+z is no greater than 2500;
L¹ is a linking group comprising a moiety formed by the chemical reaction of two click motifs;
X is, independently for each occurrence, NH, O, or S;
D represents an oligomer or copolymer (block copolymer or random copolymer) defined by the formula below

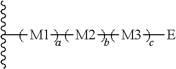

wherein M1 comprises a repeat unit comprising a hydrophilic sidechain;
wherein M2 comprises a repeat unit comprises a cholesterol-containing sidechain;
wherein M3 comprises a repeat unit comprising a hydrophilic sidechain;
a is an integer from 0 to 500;
b is an integer from 2 to 500;
c is an integer from 0 to 500;
E is absent, or represents a terminal unit optionally selected from the group consisting of dithioester, trithiocarbonate, xanthate;
L⁶ is absent, or represents a linking group; and
CM1 represents a first click motif.

In some embodiments, CM1 can comprise an azide.

In some embodiments, a can be 0. In other embodiments, a can be at least 1 (e.g., at least 5, at least 10, at least 25, at least 50, at least 75, at least 100, at least 150, at least 200, at least 250, at least 300, at least 350, at least 400, or at least 450). In some embodiments, a can be 500 or less (e.g., 450 or less, 400 or less, 350 or less, 300 or less, 250 or less, 200 or less, 150 or less, 100 or less, 75 or less, 50 or less, 25 or less, 10 or less, or 5 or less).

a can be from any of the minimum values described above to any of the maximum values described above. For example, in some embodiments, a can be from 0 to 500 (e.g., from 0 to 100, from 1 to 500, or from 1 to 100).

In other embodiments, b can be at least 2 (e.g., at least 5, at least 10, at least 25, at least 50, at least 75, at least 100, at least 150, at least 200, at least 250, at least 300, at least 350, at least 400, or at least 450). In some embodiments, b can be 500 or less (e.g., 450 or less, 400 or less, 350 or less, 300 or less, 250 or less, 200 or less, 150 or less, 100 or less, 75 or less, 50 or less, 25 or less, or less, or 5 or less).

b can be from any of the minimum values described above to any of the maximum values described above. For example, in some embodiments, b can be from 2 to 500 (e.g., from 2 to 100).

In some embodiments, c can be 0. In other embodiments, c can be at least 1 (e.g., at least 5, at least 10, at least 25, at least 50, at least 75, at least 100, at least 150, at least 200, at least 250, at least 300, at least 350, at least 400, or at least 450). In some embodiments, c can be 500 or less (e.g., 450 or less, 400 or less, 350 or less, 300 or less, 250 or less, 200 or less, 150 or less, 100 or less, 75 or less, 50 or less, 25 or less, 10 or less, or 5 or less).

c can be from any of the minimum values described above to any of the maximum values described above. For example, in some embodiments, c can be from 0 to 500 (e.g., from 0 to 100, from 1 to 500, or from 1 to 100).

In some embodiments, at least one of a and c is from 2 to 500 (e.g., from 2 to 100).

In some embodiments, the modified hyaluronic acid can be covalently crosslinked by reaction of the copolymer defined by Formula IVA with a crosslinker defined by the structure below

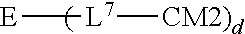

wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

In some embodiments, the click reaction can comprise a strain-promoted alkyne-azide cycloaddition (SPAAC). In some embodiments, CM2 can comprise an alkyne. In other aspects, the CM2 can comprise a dibenzocyclooctyne (DBCO) moiety.

In some embodiments, E can comprise an oligomer or polymer.

In some embodiments of Formula IVA, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IVA, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula IVA, x can be from 1 to 2400 (e.g., from 1 to 1500, from 1 to 1000, from 25 to 1000, or from 25 to 500).

In some embodiments of Formula IVA, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IVA, y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula IVA, y can be from 10 to 2400 (e.g., from 10 to 1500, from 10 to 1000, from 25 to 1000, or from 25 to 500).

In some embodiments of Formula IVA, z can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IVA, z can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

z can range from any of the minimum values described above to any of the maximum values described above. For example, in some embodiments of Formula IVA, z can be from 1 to 2400 (e.g., from 1 to 1500, from 1 to 1000, from 25 to 1000, or from 25 to 500).

In some embodiments of Formula IVA, x+y+z cm be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula IVA, x+y+z can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, or less, 15 or less, or 10 or less).

x+y+z can range from my of the minimum values described above to any of the maximum values described above. For example, in some embodiments of Formula IVA, x+y+z can be from 10 to 2400 (e.g., from 10 to 1500, from 10 to 1000, from 25 to 1000, or from 25 to 500).

### Click Chemistry and Click Motifs

The modified hyaluronic acids described above can be functionalized and/or crosslinked using click chemistry.

Click chemistry refers to a class chemical reaction (referred to as a “click reaction”) between two click groups that exhibit good yields, wide functional group tolerance, and are highly selective even in the presence of a complex mixture of biological molecules. These characteristics allow the click reactions to proceed even in vivo. Example click motif pairs used as the first click motif and the second click motif include, but not limited to, azide with phosphine; azide with cyclooctyne; nitrone with cyclooctyne; nitrile oxide with norbornene; oxanorbornadiene with azide; trans-cyclooctene with s-tetrazine; quadricyclane with bis(dithiobenzil)nickel(II).

In some embodiments, the second click motif comprises an alkene, e.g., a cyclooctene, e.g., a transcyclooctene (TCO) or norbornene (NOR), and the first click motif comprises a tetrazine (Tz). In other embodiments, the second click motif comprises an alkyne, e.g., a cyclooctyne such as dibenzocyclooctyne (DBCO), and the first click motif comprises an azide (Az). In some embodiments, the second click motif comprises a Tz, and the first click motif comprises an alkene such as transcyclooctene (TCO) or norbornene (NOR). Alternatively or in addition, the first click motif comprises an Az, and the second click motif comprises a cyclooctyne such as dibenzocyclooctyne (DBCO). TCO reacts specifically in a click chemistry reaction with a tetrazine (Tz) moiety. DBCO reacts specifically in a click chemistry reaction with an azide (Az) moiety. Norbornene reacts specifically in a click chemistry reaction with a tetrazine (Tz) moiety.

Exemplary click chemistry reactions (and by extension click motifs) are shown below. For example, copper(I)-catalyzed Azide-Alkyne Cycloaddition (CuAAC) comprises using a Copper (Cu) catalyst at room temperature. The Azide-Alkyne Cycloaddition is a 1,3-dipolar cycloaddition between an azide and a terminal or internal alkyne to give a 1,2,3-triazole.

Another example of click chemistry includes Staudinger ligation, which is a reaction that is based on the classic Staudinger reaction of azides with triarylphosphines. It launched the field of bioorthogonal chemistry as the first reaction with completely abiotic functional. The azide acts as a soft electrophile that prefers soft nucleophiles such as phosphines. This is in contrast to most biological nucleophiles which are typically hard nucleophiles. The reaction proceeds selectively under water-tolerant conditions to produce a stable product. Phosphines are completely absent from living systems and do not reduce disulfide bonds despite mild reduction potential. Azides had been shown to be biocompatible in FDA-approved drugs such as azidothymidine and through other uses as cross linkers. Additionally, their small size allows them to be easily incorporated into biomolecules through cellular metabolic pathways.

Copper-free click chemistry is a bioorthogonal reaction first developed by Carolyn Bertozzi as an activated variant of an azide alkyne cycloaddition. Unlike CuAAC, Cu-free click chemistry has been modified to be bioorthogonal by eliminating a cytotoxic copper catalyst, allowing reaction to proceed quickly and without live cell toxicity. Instead of copper, the reaction is a strain-promoted alkyne-azide cycloaddition (SPAAC). It was developed as a faster alternative to the Staudinger ligation, with the first generations reacting over sixty times faster. The incredible bioorthogonality of the reaction has allowed the Cu-free click reaction to be applied within cultured cells, live zebrafish, and mice. Cyclooctynes were selected as the smallest stable alkyne ring which increases reactivity through ring strain which has calculated to be 19.9 kcal/mol.

Copper-free click chemistry also includes nitrone dipole cycloaddition. Copper-free click chemistry has been adapted to use nitrones as the 1,3-dipole rather than azides and has been used in the modification of peptides.

This cycloaddition between a nitrone and a cyclooctyne forms N-alkylated isoxazolines. The reaction rate is enhanced by water and is extremely fast with second order rate constants ranging from 12 to 32 M⁻¹ s⁻¹, depending on the substitution of the nitrone. Although the reaction is extremely fast, incorporating the nitrone into biomolecules through metabolic labeling has only been achieved through post-translational peptide modification.

Another example of click chemistry includes norbornene cycloaddition. 1,3 dipolar cycloadditions have been developed as a bioorthogonal reaction using a nitrile oxide as a 1,3-dipole and a norbornene as a dipolarophile. Its primary use has been in labeling DNA and RNA in automated oligonucleotide synthesizers.

Norbornenes were selected as dipolarophiles due to their balance between strain-promoted reactivity and stability. The drawbacks of this reaction include the cross-reactivity of the nitrile oxide due to strong electrophilicity and slow reaction kinetics.

Another example of click chemistry includes oxanorbornadiene cycloaddition. The oxanorbornadiene cycloaddition is a 1,3-dipolar cycloaddition followed by a retro-Diels Alder reaction to generate a triazole-linked conjugate with the elimination of a furan molecule. This reaction is useful in peptide labeling experiments, and it has also been used in the generation of SPECT imaging compounds.

Ring strain and electron deficiency in the oxanorbornadiene increase reactivity towards the cycloaddition rate-limiting step. The retro-Diels Alder reaction occurs quickly afterwards to form the stable 1,2,3 triazole. Limitations of this reaction include poor tolerance for substituents which may change electronics of the oxanorbornadiene and low rates (second order rate constants on the order of 10⁻⁴).

Another example of click chemistry includes tetrazine ligation. The tetrazine ligation is the reaction of a trans-cyclooctene and an s-tetrazine in an inverse-demand Diels Alder reaction followed by a retro-Diels Alder reaction to eliminate nitrogen gas. The reaction is extremely rapid with a second order rate constant of 2000 M⁻¹-s⁻¹ (in 9:1 methanol/water) allowing modifications of biomolecules at extremely low concentrations.

The highly strained trans-cyclooctene is used as a reactive dienophile. The diene is a 3,6-diaryl-s-tetrazine which has been substituted in order to resist immediate reaction with water. The reaction proceeds through an initial cycloaddition followed by a reverse Diels Alder to eliminate N₂ and prevent reversibility of the reaction.

Not only is the reaction tolerant of water, but it has been found that the rate increases in aqueous media. Reactions have also been performed using norbornenes as dienophiles at second order rates on the order of 1 M⁻¹·s⁻¹ in aqueous media. The reaction has been applied in labeling live cells and polymer coupling.

Another example of click chemistry includes is [4+1]cycloaddition. This isocyanide click reaction is a [4+1]cycloaddition followed by a retro-Diels Alder elimination of N₂.

The reaction proceeds with an initial [4+1]cycloaddition followed by a reversion to eliminate a thermodynamic sink and prevent reversibility. This product is stable if a tertiary amine or isocyanopropanoate is used. If a secondary or primary isocyanide is used, the produce will form an imine which is quickly hydrolyzed.

Isocyanide is a favored chemical reporter due to its small size, stability, non-toxicity, and absence in mammalian systems. However, the reaction is slow, with second order rate constants on the order of 10⁻² M⁻¹·s⁻¹.

Another example of click chemistry includes quadricyclane ligation. The quadricyclane ligation utilizes a highly strained quadricyclane to undergo [2+2+2]cycloaddition with n systems.

Quadricyclane is abiotic, unreactive with biomolecules (due to complete saturation), relatively small, and highly strained (^{˜}80 kcal/mol). However, it is highly stable at room temperature and in aqueous conditions at physiological pH. It is selectively able to react with electron-poor π systems but not simple alkenes, alkynes, or cyclooctynes.

Bis(dithiobenzil)nickel(II) was chosen as a reaction partner out of a candidate screen based on reactivity. To prevent light-induced reversion to norbornadiene, diethyldithiocarbamate is added to chelate the nickel in the product.

These reactions are enhanced by aqueous conditions with a second order rate constant of 0.25 M⁻¹·s⁻¹. Of particular interest is that it has been proven to be bioorthogonal to both oxime formation and copper-free click chemistry.

The exemplary click chemistry reactions have high specificity, efficient kinetics, and occur in vivo under physiological conditions. See, e.g., Baskin et al. Proc. Natl. Acad. Sci. USA 104 (2007):16793; Oneto et al. Acta biomaterilia (2014); Neves et al. Bioconjugate chemistry 24 (2013):934; Koo et al. Angewandte Chemie 51 (2012):11836; and Rossin et al. Angewandte Chemie 49 (2010):3375. For a review of a wide variety of click chemistry reactions and their methodologies, see e.g., Nwe K and Brechbiel M W, 2009 Cancer Biotherapy and Radiopharmaceuticals. 24(3): 289-302; Kolb H C et al., 2001 Angew. Chem. Int. Ed. 40: 2004-2021. The entire contents of each of the foregoing references are incorporated herein by reference.

Exemplary click motif pairs are shown in the table below. Functional groups formed by reaction of click motif pairs are well known in the art.

| Functional | | | |
|---|---|---|---|
| group/Click | Paired | | Reaction type |
| Motif | with | Functional group/Click Motif | (Reference) |
| azide | | Phosphine | Staudinger ligation |
| | | | (Saxon et al. Science |
| | | | 287(2000):2007-10) |
| azide | | Cyclooctyne, e.g., dibenzocyclooctyne, one of | Copper-free click |
| | | the cyclooctynes shown below, or other similar | chemistry (Jewett et al. J. |
| | | cyclooctynes: | Am. Chem. Soc. |
| | | 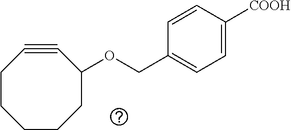 [SMILES: O=C(O)c1ccc(COC2C#CCCCCC2)cc1] | 132.11(2010):3688-90; Sletten et al. Organic Letters 10.14 (2008):3097-9; Lutz. Angew. Chem., Int. Ed 47.12(2008):2182) |
| | | 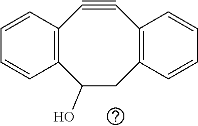 [SMILES: OC1Cc2ccccc2C#Cc2ccccc21] | |
| | | 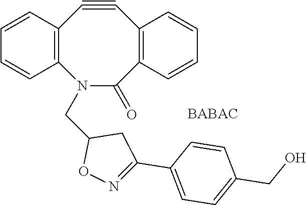 [SMILES: O=C1c2ccccc2C#Cc2ccccc2N1CC1CC(c2ccc(CO)cc2)=NO1] | |
| | | 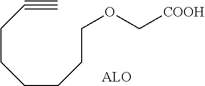 | |
| | | 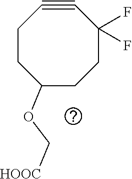 [SMILES: O=C(O)COC1CCC#CC(F)(F)CC1] | |
| | | 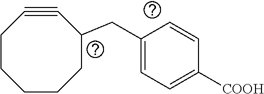 [SMILES: O=C(O)c1ccc(CC2C#CCCCCC2)cc1] | |
| | | 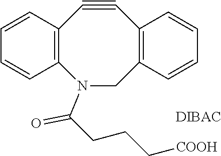 [SMILES: O=C(O)CCCC(=O)N1Cc2ccccc2C#Cc2ccccc21] | |
| | | 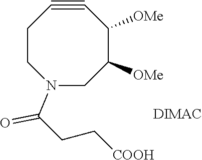 [SMILES: CO[C@H]1C#CCCN(C(=O)CCC(=O)O)C[C@@H]1OC] | |
| nitrone | | Cyclooctyne | Nitrone Dipole |
| | | | Cycloaddition (Ning et |
| | | | al. Angew. Chem., Int. Ed |
| | | | 49.17 (2010):3065) |
| Nitrile oxide | | Norbornene | Norbornene |
| | | | Cycloaddition |
| | | | (Gutsmiedl et al. Organic |
| | | | Letters |
| | | | 11.11(2009):2405-8) |
| oxanorbornadiene | | Azide | Oxanorbornadiene |
| | | | Cycloaddition (Van |
| | | | Berkel et al. |
| | | | ChemBioChem |
| | | | 8.13(2007):1504-8) |
| Trans-cyclooctene, | | s-tetrazine | Tetrazine ligation |
| norbornene, or | | | (Hansell et al. J. Am. |
| other alkene | | | Chem. Soc. |
| | | | 133.35(2011): 13828-31) |
| nitrile | | 1,2,4,5-tetrazine | [4 + 1] cycloaddition |
| | | | (Stackman et al. Organic |
| | | | and Biomol. Chem. |
| | | | 9.21(2011):7303) |
| quadricyclanc | | Bis(dithiobenzil)nickel(II) | Quadricyclanc Ligation |
| | | | (Sletten et al. J. Am. |
| | | | Chem. Soc. |
| | | | 133.44(2011):17570-3) |
| Ketone or aldehyde | | Hydrazines, hydrazones, oximes, | Non-aldol carbonyl |
| | | amines, ureas, thioureas, etc. | chemistry |
| | | | (Khomyakova EA, et al. |
| | | | Nucleosides Nucleotides |
| | | | Nucleic Acids. 30(7-8) |
| | | | (2011) 577-84 |
| Thiol | | Maleimide | Michael addition |
| | | | (Zhou et al. Bioconjug |
| | | | Chem 2007 18(2):323- |
| | | | 32.) |
| Dienes | | Dienophiles | Diels Alder (Rossin et al. |
| | | | Nucl Med. (2013) |
| | | | 54(11): 1989-95) |
| Tetrazine | | norbornene, propene, trans-cyclooctene, | |
| | | other strained alkenes. | |

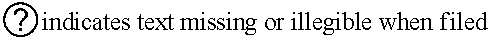

Other suitable include the motifs can be found, for example, in Patterson, D. M., et al. “Finding the Right (Bioorthogonal) Chemistry,” ACS Chem. Biol., 2014, 9(3): 592-605; Akgun, B., et al. “Synergic “Click” Boronate/Thiosemicarbazone System for Fast and Irreversible Bioorthogonal Conjugation in Live Cells,” J. Am. Chem. Soc., 2017, 139(40): 14285-14291; and Akgun, B. and Hall, D. G. “Fast and Tight Boronate Formation for Click Bioorthogonal Conjugation,” Angew. Chem., Int. Ed. 2016, 55(12): 3909-3913, each of which is hereby incorporated by reference in its entirety.

### Methods of Making

The modified hyaluronic acids can be prepared using synthetic procedures known in the art. By way of exemplification, a few representative syntheses are described below.

HA with molecular weight of 50, 100, 200, 500, or 1000 kD can be conjugated with 1-azido-3-aminopropane via NHS/EDC chemistry to prepare HA azide with DS ranging from 0.1 to 0.2. Isolated cholesterol-DBCO can be obtained through two-step synthesis, including: (1) the synthesis of cholesterol amine from cholesterol chloroformate and ethylenediamine; and (2) the synthesis of cholesteryl DBCO using cholesterol amine and DBCO NHS ester. Cholesterol oligomers with the degree of polymerization (DP) from 2 to 50 can be obtained through the reversible addition-fragmentation chain transfer (RAFT) polymerization using a DBCO-coupled chain transfer agent. Isolated cholesterol and cholesterol oligomers bearing a DBCO end groups can be readily conjugated with HA azide through SPAAC click chemistry. The reaction can be carried out in any suitable solvent, such as DMSO or a DMSO aqueous solution. Different amounts of cholesterol can be added respect to the azide content of HA to obtain the cholesterol-modified HA with DS of cholesterol from 0.001 to 0.1.

Suitable methods for preparing the modified hyaluronic acids are further detailed in the Examples below.

### Nanoparticles, Microparticles, and Hydrogels

Also provided herein are particles (e.g., nanoparticles and microparticles) formed from the modified hyaluronic acid polymers described herein. In some embodiments, the particles can further comprise an active agent encapsulated within the particles.

Also provided herein are hydrogels comprising the modified hyaluronic acid polymers described herein. In some aspects, the hydrogel can further comprise an active agent dispersed within the hydrogel. In some aspects, the hydrogel can further comprise one or more cells disposed on or within the hydrogel.

Both hydrophobic and hydrophilic active agents can be incorporated in the particles and hydrogels described herein. Exemplary classes of active agents include steroids, growth factors, anti-proliferative agents, and antibiotics. Generally, the particles and hydrogels can include from about 0.01% by weight to about 20% by weight active agent, depending on its potency. Illustrative amounts of bioactive agent contained in the hydrogel (based on overall wet gel weight) are from about 10% to about 20% by weight, e.g., for a less potent active agent, and from about 0.01% to about 10% by weight, or from about 0.01% to about 5%, or from about 0.01% to about 3%, or from about 0.1 to about 2% active agent, or even from about 0.1 to about 1% active agent, e.g., for a more potent active agent.

Advantageously, the particles and hydrogels can be formed both under mild reaction conditions and can be formed in the absence of a polymerization initiator. Moreover, sufficient gelation occurs in the absence of the application of an external energy source. For example, the gel-formation reaction can be carried out at a temperature ranging from about 20° C. to 45° C.—and in the absence of initiators and accelerants. Additionally, the gelation, i.e., hydrogel formation, occurs without the release of any small molecule chemical by-products. Thus, the particles and hydrogels provided herein contain a minimal number of additives or contaminants that could potentially lead to a pro-inflammatory response upon in-vivo administration.

Sterile particles and hydrogels can be formed under sterile conditions, e.g., by placing aqueous solutions of each of the modified hyaluronic acid and crosslinker into a sterile syringe and or centrifuge tube, followed by thorough mixing.

If desired, additional unmodified hyaluronic acid, typically in the form of an aqueous solution or mixture, may optionally be added to either the hydrogel precursor formulation, prior to gel formation, or after gel formation (e.g., to a gel slurry), to provide a composition comprising hydrogel particles in an aqueous solution of hyaluronic acid.

An active agent may be added to the reaction mixture prior to crosslinking or alternatively, added to the crosslinked gel after formation. Alternatively, living cells such as stem cells, parenchimal stem cells, blood derived cells, and bone marrow cells can be incorporated into the subject hydrogels. These concepts are illustrated in the Examples below.

The pH of compositions comprising the particles and hydrogels and subject hydrogel/polymer solution dispersions can be modified by the addition of buffers, acid and bases. The preferred pH range for the subject particles and hydrogels and subject hydrogel/polymer solution dispersions is from about 5-8 and more preferably from about 6-7.6.

The ionic strength of the particles and hydrogels and subject hydrogel/polymer solution dispersions can be modified by the addition of salts. One preferred salt used to modify the ionic strength of the particles and hydrogels and subject hydrogel/polymer solution dispersions is sodium chloride. A preferred final ionic strength of the particles and hydrogels and subject hydrogel/polymer solution dispersions is selected such that the particles and hydrogels and subject hydrogel/polymer solution dispersions are about isotonic.

Pharmaceutically acceptable preservatives may also be added to the particles and hydrogels and subject hydrogel/polymer solution dispersions. These can include agents such as sodium benzoate or benzyl alcohol.

The particles and hydrogels and subject hydrogel/polymer solution dispersions may, in certain embodiments, be packaged in a syringe. The syringe can be made from plastic (e.g. polypropylene, polycarbonate, polystyrene) or glass or any other pharmaceutically acceptable material. The volume of the particles and hydrogels and subject hydrogel/polymer solution dispersions contained within the syringe may range from 0.5 mL to 20 mL with preferable volumes being 1 mL, 2 mL, 3 mL, 4 mL, 5 mL, 6 mL and 7 mL.

The hydrogel material may be processed into particles having a size ranging from about 0.10 to 3.0 millimeters, or may be in the form of an aqueous gel slurry. For example, gelled material can be broken up into pieces, mixed with saline, and allowed to swell. Appropriately sized particles can then be formed from the gel material by extrusion through a mesh having the desired screen size, e.g., from about 0.10 to 3.0 millimeters. The resulting particles, when placed in an aqueous medium, form a gel slurry. In one embodiment, the gel is packaged in a syringe suitable for use with a 18-21 gauge needle, such that the hydrogel can be injected, i.e., into an intra-articular space. Generally, the volume of hydrogel composition injected into an intra-articular space of a subject ranges from about 0.5 to about 8 mL, preferably from about 3 to 6 mL, or even from about 4-6 mL.

In some embodiments, the particles and hydrogels can be provided as sterile compositions. As described above, the particles and hydrogels may be provided in a sealed container such as a syringe (which can be capped, optionally with a vented cap). The syringe may then be placed in a container, such as a foil pouch which is then sealed. The pouch may be vacuum sealed, sealed under an inert gas such as nitrogen or argon, or sealed following one or more vacuum/back fill cycles where the back fill gas is an inert gas such as nitrogen or argon. For the pouch sealed under one or more vacuum/back fill cycles, the cycle can be adjusted such that the pouch is finally sealed under either vacuum or an inert gas. The pouch may optionally contain a dessicant and/or an oxygen scavenger.

### Active Agents

The particles, hydrogels, hydrogel precursors, and related compositions and/or kits provided herein may optionally comprise an active agent.

The active agent can comprise any suitable active agent, such as a small molecule therapeutic agent, a cosmetic agent, a diagnostic agent (e.g., detectable label), a native or synthetic polymer, a protein, a polypeptide, an oligonucleotide, antimicrobial particles (e.g., metal particles such as silver nanoparticles), minerals, a bioceramic, and/or a cell.

Examples of active agents that can be included in the compositions and combinations provided herein include antimicrobials, antibiotics, analgesics, antibiotics, antiproliferative/antimitotic agents including natural products such as vinca alkaloids (e.g. vinblastine, vincristine, and vinorelbine), paclitaxel, epidipodophyllotoxins (e.g. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase); antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine [cladribine]); platinum coordination complexes (cisplatin, carboplatin), procarbazine, hydroxyurea, mitotane, aminoglutethimide; hormones (e.g. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (such as brefeldin A); anti-inflammatory agents such as adrenocortical steroids (hydrocortisone, hydrocortisone acetate, cortisone acetate, tixocortol pivalate, prednisolone, methylprednisolone, prednisone, triamcinolone acetonide (or any other pharmaceutically acceptable salts of triamcinolone), triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, and fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate. Beclomethasone dipropionate monohydrate, flunisolide, fluticasone propionate, mometasone furoate monohydrate, triamcinolone acetonide, fluticasone, furoate, non-steroidal agents (salicylic acid derivatives e.g. aspirin); para-aminophenol derivatives, i.e. acetaminophen; indole and indene acetic acids (indomethacin, sulindac, and etodolac), heteroaryl acetic acids (tolmetin, diclofenac, and ketorolac), arylpropionic acids (ibuprofen and derivatives), anthranilic acids (mefenamic acid and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressive (cyclosporine, tacrolimus (FK-506), sirolimus (rapamycin), azathioprine, mycophenolate mofetil); mitogenic or morphogenic growth factor proteins, peptides or mimetics; vascular endothelial growth factor (VEGF), fibroblast growth factor (FGF), transforming growth factor-β (TGF-β) superfamily members including TGF-β's and bone morphogenic proteins (BMP's) such as BMP-2, 3, 4, 5, 6, 7, 8; insulin and insulin-like growth factors (IGF's), hepatocyte growth factor (HGF), epidermal growth factors (EGF's), Hedgehog proteins (SHH and IHH), activins, inhibins, demineralized bone (DBM) and platelet-derived growth factors (PDGF's), hematopoietic growth factors (G-CSF, CSF-1, GM-CSF, erythropoietin, cytokines and lymphokines including the interleukin family (IL-1 to 34)), interferons, nerve growth factors (NGF's), neutralizing, antagonist or agonist antibodies, growth factor receptor agonists or antagonists, nitric oxide donors; anti-sense oligonucleotides, transcription factors, signaling cascade mediators, and combinations thereof.

Antibiotics include antibiotics of the lincomycin family (referring to a class of antibiotic agents originally recovered from Streptomyces lincolnensis); antibiotics of the tetracycline family (referring to a class of antibiotic agents originally recovered from Streptomyces aureofaciens); sulfur-based antibiotics such as the sulfonamides; and so forth. Exemplary antibiotics of the lincomycin family include lincomycin itself (6,8-dideoxy-6-[[(1-methyl-4-propyl-2-pyrrolidinyl)-carbonyl]amino-]-1-thio-L-threo- [symbol] -D-galacto-octopyranoside), clindamycin, the 7-deoxy, 7-chloro derivative of lincomycin (e.g., 7-chloro-6,7,8-trideoxy-6-[[(1-methyl-4-propyl-2-pyrrolidinyl) carbonyl]amino]-1-thio-L-threo- [symbol] -D-galacto-octopyranoside), and pharmacologically acceptable salts and esters thereof. Exemplary antibiotics of the tetracycline family include tetracycline itself 4-(dimethylamino)-1,4,4α,5,5α,6,11,12α-octahydro-3,6,12,12α-pentahydroxy-6-methyl-1,11-dioxo-2-naphthacenecarboxamide), chlortetracycline, oxytetracycline, tetracycline, demeclocycline, rolitetracycline, methacycline and doxycycline and their pharmaceutically acceptable salts and esters, particularly acid addition salts such as the hydrochloride salt. Exemplary sulfur-based antibiotics include, but are not limited to, the sulfonamides sulfacetamide, sulfabenzamide, sulfadiazine, sulfadoxine, sulfamerazine, sulfamethazine, sulfamethizole, sulfamethoxazole, and pharmacologically acceptable salts and esters thereof, e.g., sulfacetamide sodium. Antimicrobials and/or antibiotics further include compounds such as erythromycin, bacitracin, neomycin, penicillin, polymyxin B, tetracyclines, viomycin, chloromycetin and streptomycins, cefazolin, ampicillin, azactam, tobramycin, clindamycin and gentamycin.

Analgesics include compounds such as lidocaine, benzocaine, and marcaine.

Examples of cosmetics include, for example, anti-aging formulations including anti-oxidants, vitamins, nutraceuticals, natural products, and other components.

Examples of antimicrobial particles include, for example, antimicrobial metal particles (e.g., silver particles and/or copper particles). In certain examples, the antimicrobial particles can comprise silver nanoparticles and/or copper nanoparticles.

Examples of hydroxyapatite, P-tricalcium phosphate, tetracalcium phosphate, biphasic calcium phosphate, nanocrystalline hydroxyapatite, bioactive glass, 45S5 bioactive glass, titanium oxide, and/or aluminum oxide.

The particles, hydrogels, hydrogel precursors, and related compositions and/or kits provided herein may also include living cells. Exemplary living cells include stem cells, parenchimal stem cells, blood-derived cells, and bone marrow cells.

In some embodiments, the particles, hydrogels, hydrogel precursors, and related compositions and/or kits provided herein can comprise a corticosteroid. Examples of suitable corticosteroids include hydrocortisone, hydrocortisone acetate, cortisone acetate, tixocortol pivalate, prednisolone, methylprednisolone, prednisone, triamcinolone, triamcinolone salts such as triamcinolone acetonide, triamcinolone benetonide, triamcinolone furetonide, triamcinolone hexacetonide, triamcinolone diacetate, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, beclomethasone dipropionate monohydrate, flunisolide, fluticasone propionate, mometasone furoate monohydrate, and fluticasone furoate.

The active agent can be admixed, suspended in, or entrapped within the particles and hydrogels as provided herein. Alternatively, the active agent may be in the form of a polymer conjugate, or, may be covalently attached, in a releasable fashion, to a component used to prepare the hydrogel, e.g., the modified hyaluronic acid or crosslinker.

### Methods of Use

Particles and hydrogels that comprise active agent (e.g., pharmaceuticals or other bioactive moieties) are particularly useful in drug delivery applications, for example, as depots for sustained release, controlled release, or slow release of active agents.

The particles and hydrogels described herein can also be used in a variety of other applications, including as a scaffolding material for tissue engineering, for wound or fracture healing (either by itself, or as a substrate for cell delivery, e.g. the delivery of chondrocytes for repairing cartilage damage), joint damage, and cosmetic applications.

In some embodiments, the particle and hydrogel compositions described herein can exhibit reduced undesirable side effects on the cartilage in comparison to commercially available viscosupplements. In embodiments in which the particle and hydrogel further comprises an active agent, the particles and hydrogels can exhibit reduced undesirable side effects on the cartilage when compared to administration of an equivalent amount of active agent absent particle or hydrogel incorporation.

The particle and hydrogel compositions described herein may be used in injectable or implantable formulations, for use, e.g., embryonic development, tissue organization, wound healing, angiogenesis and tumorigenesis. By way of example, particle and hydrogel compositions comprising a corticosteroid can be useful for providing relief of pain and/or inflammation experienced by a subject. Injection of a therapeutically effective amount of the particle or hydrogel composition into an intra-articular space of a joint can be effective, e.g., for providing sustained relief of joint pain experienced by a subject.

The particle and hydrogel compositions provided herein, optionally containing one or more active agents, may also be used as adhesive compositions, e.g., as tissue adhesives and sealants that may be used for various applications, including preventing bleeding, covering open wounds, and other biomedical applications. These compositions may be used in, for example, apposing surgically incised or traumatically lacerated tissues, retarding blood flow such as those from wounds, preventing restenosis or blood clotting, drug delivery; dressing burns, and aiding repair and regrowth of living tissue. The hyaluronic acid-based polymer composition as provided herein may be used for supplementing or inducing and regenerating damaged organs or tissues in a mammalian subject, such as a human. The composition is decomposed or absorbed, or alternatively, remains in the subject (e.g., mammalian subject) without having adverse influences on subject when embedded or contained therein.

The subject compositions may be used as tissue fillers, dermal fillers, bulking agents, and embolic agents as well as agents to repair cartilage defects/injuries and osteoconductive or osteoinductive agents including growth factors, minerals and bioceramics to enhance bone repair and/or growth.

The subject compositions may also be used in the treatment of osteoarthritis or rheumatoid arthritis, or for other inflammatory arthritis such as gout or calcium pyrophosphate deposition disease (e.g., by injection into the intra-articular space of a joint), or in the reduction or prevention of adhesions that can form following a surgical procedure.

For particles and hydrogels comprising an active agent, such compositions may be used as delivery systems for the treatment of conditions such as osteoarthritis, sinusitis, allergic rhinitis and chronic rhinosinusitis, among others. Such compositions may also be used as dermal fillers, agents to repair cartilage defects/injuries and agents to enhance bone repair and/or growth.

The subject compositions can also be employed for the encapsulation of cells and tissues (e.g., for pancreatic islet cell encapsulation).

The subject compositions can also be employed as scaffolds for tissue growth and tissue engineering.

The present application will now be described in connection with certain embodiments, which are not intended to limit the scope of the invention. On the contrary, the present application covers all alternatives, modifications, and equivalents as included within the scope of the claims. Thus, the following will illustrate the practice of the present application, for the purposes of illustration of certain embodiments and is presented to provide what is believed to be a useful and readily understood description of its procedures and conceptual aspects.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Synthesis and Characterization of Cholesterol-Modified Hyaluronic Acids

An example synthetic route for the functionalization of HA is depicted in Scheme 1.

¹H NMR spectroscopy was performed on a Bruker AVANCE III HD 500 MHz spectrometer at 25° C. The Fourier transformed infrared (FTIR) spectra was recorded on a Nicolet IR 100 spectrometer (Termo Electron Corporation).

### Scheme 1. Synthetic Route for Cholesteryl HA (Chol HA).

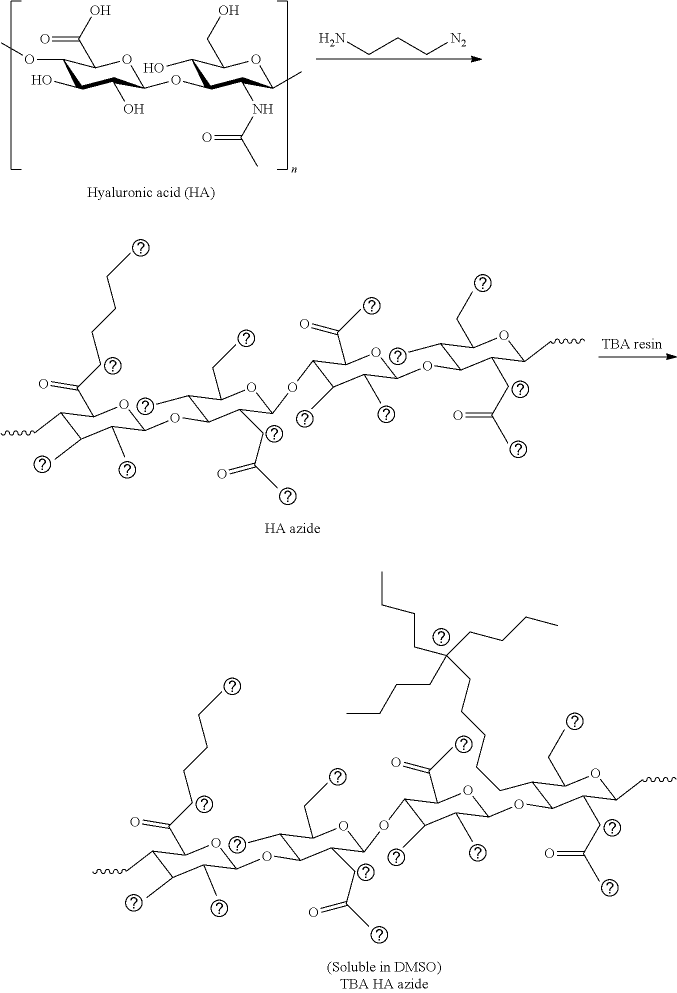

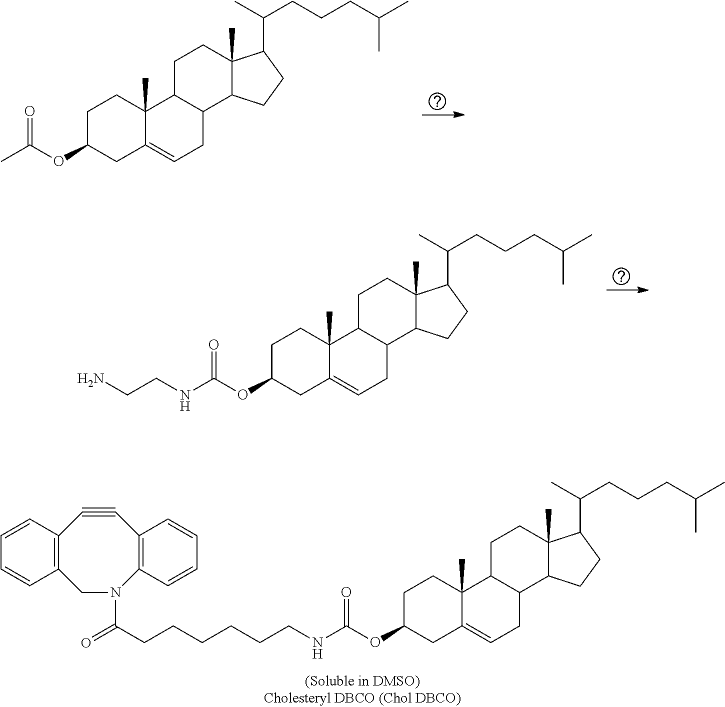 [SMILES: CC(=O)O[C@H]1CC[C@@]2(C)C(=CCC3C4CCC(C(C)CCCC(C)C)[C@@]4(C)CCC32)C1 ; CC(C)CCCC(C)C1CCC2C3CC=C4C[C@@H](OC(=O)NCCN)CC[C@]4(C)C3CC[C@]12C ; CC(C)CCCC(C)C1CCC2C3CC=C4C[C@@H](OC(=O)NCCCCCCC(=O)N5Cc6ccccc6C#Cc6ccccc65)CC[C@]4(C)C3CC[C@]12C]

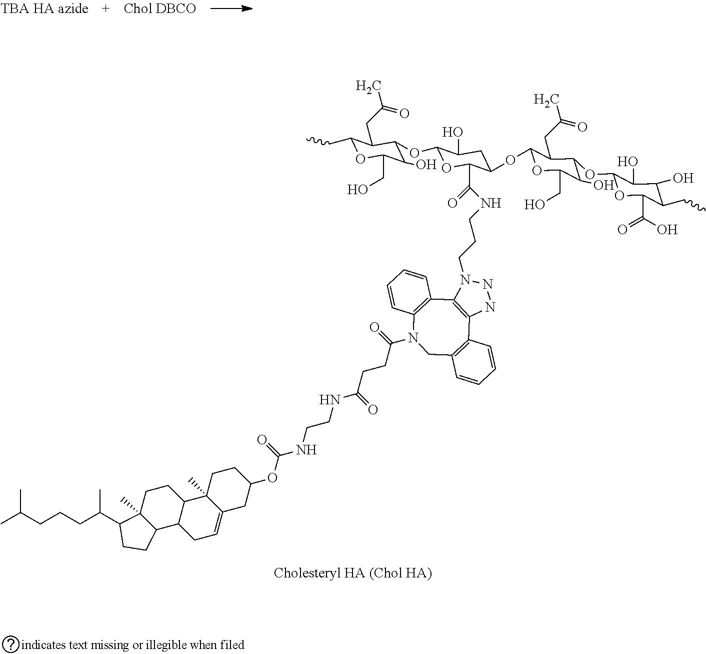

### Synthesis of 1-azido-3-aminopropane

3-chloropropylamine hydrochloride (2 g, 15 mmol) and sodium azide (3 g, 46 mmol) were dissolved in 15 mL of distilled water. Reaction was carried out at 80° C. for 15 hr before the aqueous mixture was concentrated, followed by cooling down the concentrated solution in an ice bath for 5 min. Diethyl ether (25 mL) was added and mixed for another 5 min before potassium hydroxide (2 g) was added and stirred in an ice bath for 10 min. The organic layer was separated and collected. The aqueous layer was extracted with diethyl ether (10 mL) two more times. The combined diethyl ether fractions were dried over sodium sulfate before being concentrated using a rotatory evaporator at RT. The product was obtained as a light green liquid (Yield: 85%). Synthesis was confirmed by ¹H NMR (CDCl₃).

### Synthesis of Hyaluronic Aid Azide (HA azide) Sodium Salt

Following an EDC/NHS amidation procedure, hyaluronic acid (HA, 200 mg, 0.5 mmol) was dissolved in 20 mL of MES buffer (50 mM, containing 0.5 M NaCl, pH 6.5), to which EDC (192 mg, 1 mmol) was added, followed by NHS (58 mg, 0.5 mmol). The mixture was stirred for 10 min before 1-azido-3-aminopropane (50 mg, 0.5 mmol), pre-mixed with 1 mL of MES buffer, was added. The reaction was carried out for 24 hr at RT. NaCl (3.4 g) was added to screen out the charge of HA before the aqueous mixture was precipitated with ethanol (9 mL). The precipitate obtained after centrifugation (2000 rpm, 5 min) was redissolved in DI water for dialysis (MWCO 12-14 kD), with 4-5 fresh water changes. Cotton-like solid was obtained after lyophilization.

### Synthesis of Hyaluronic Acid Azide TBA Salt (TBA HA Azide)

To solubilize HA azide in DMSO, tetrabutylammonium hydroxide (TBA-OH) resins were prepared to replace the sodium cations in the HA azide sodium salt with TBA cations.

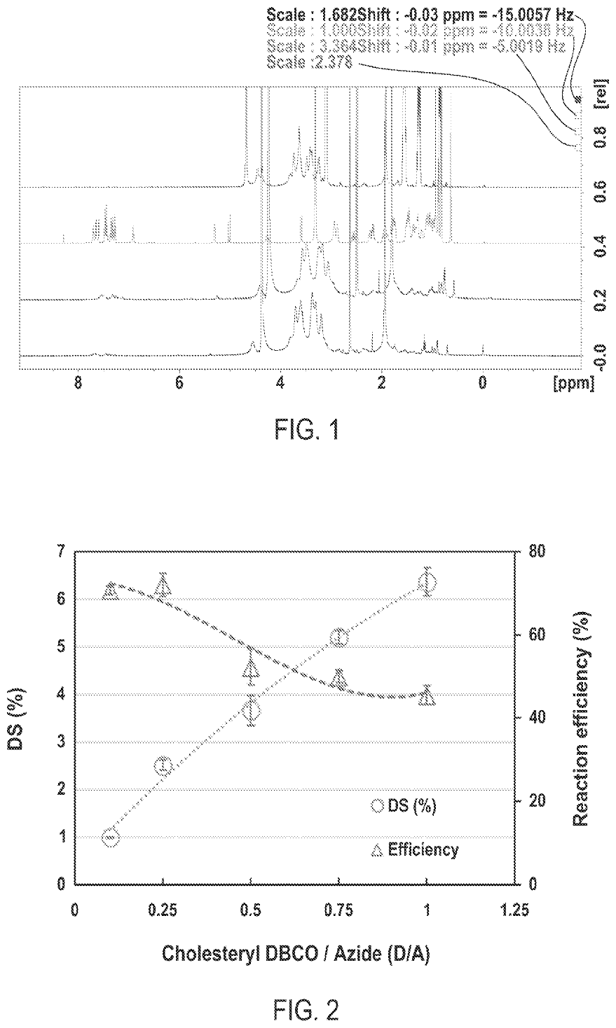

In brief, ionic exchange resin Dowex 50WX2-200H was washed with DI water before being mixed with 40% TBA-OH aqueous solution overnight. Excess TBA-OH was washed out with DI water until the pH was around 6-7. The resulting TBA resins were transferred into aqueous HA azide solution (1%) and allowed to mix for 24 hr. TBA HA azide was obtained after filtration and lyophilization. Degree of substitution of azide and TBA was confirmed as 0.14 and 0.65-0.70, respectively, according to ¹H NMR in D₂O (FIG. 1, trace (a)).

### Synthesis of Cholesteryl Amine

Cholesteryl chloroformate (100 mg, 0.22 mmol) and ethylenediamine (134 mg, 2.2 mmol) were separately dissolved in dry dichloromethane (10 mL). The cholesteryl chloroformate solution was added dropwise into the ethylenediamine solution using a syringe over 10-15 min under stirring and argon protection. The reaction mixture was stirred under argon for 1 hr at RT. Upon the completion of reaction, the mixture was washed with DI water (20 mL) twice and then brine (20 mL). The organic layer was dried over sodium sulfate before it was concentrated and purified by flash column chromatography with chloroform/methanol 4:1 as an eluent. The purified product was obtained as a white solid (yield: 67%) and stored in chloroform at −20° C. before use. Synthesis was confirmed by ¹H NMR (CDCl₃).

### Synthesis of Cholesteryl DBCO

Cholesteryl amine (50 mg, 0.1 mmol) was dissolved in 3 mL of dry dichloromethane and mixed with triethylamine (60 μL, 0.4 mmol). DBCO NHS ester (34 mg, 0.085 mmol), dissolved in 3 mL of dry dichloromethane, was added to the cholesteryl amine solution using a syringe under stirring and the reaction was carried out for 4 hr at RT in argon atmosphere. Upon removal of the solvent, the product was purified by flash column chromatography with chloroform/methanol 10:1 as an eluent. The purified product was obtained as an oil-like yellow liquid (yield: 99%). ¹H NMR (CDCl₃, 500 Hz) δ ppm: 7.66-7.60 (d, 1H), 7.44-7.38 (m, 1H), 7.37-7.16 (m, 6H), 6.00 (m, 0.88H), 5.34-5.27 (m, 1H), 5.16 (m, 0.7H), 5.12-5.05 (d, 1H), 4.43 (m, 1H), 3.64-3.57 (d, 1H), 3.30-2.90 (m, 4H), 2.80-2.67 (m, 1H), 2.36-2.17 (m, 3H), 2.17-2.07 (m, 1H), 2.00-1.70 (m, 6H), 1.70-0.73 (m, 33H), 0.60 (s, 3H) (FIG. 1, trace (b)). The purified cholesteryl DBCO was stored in DMSO (10 mg/mL) at −20° C. before use.

### Synthesis of Cholesteryl Hyaluronic Acid (Chol HA)

TBA HA azide was fully dissolved in DMSO. The volumes of stock DMSO solutions of cholesterol DBCO and TBA HA azide were adjusted to alter the ratio of DBCO to azide (D/A) from 0.1 to 1.0 for the Click reaction, which was carried out for 1 hr at RT. Equal volume of water was added to help transfer the mixture into a dialysis bag (MWCO 12-14 kD). Dialysis was performed in 10% NaCl aqueous solution for 1 day with NaCl solution changes twice, then in DI water with water change 4 times. Chol HA was lyophilized and stored in a desiccator. The degree of substitution (DS) of cholesterol in Chol HA was determined by ¹H NMR (DMSO-d6/D₂O 2:1) (FIGS. 1, traces c & d).

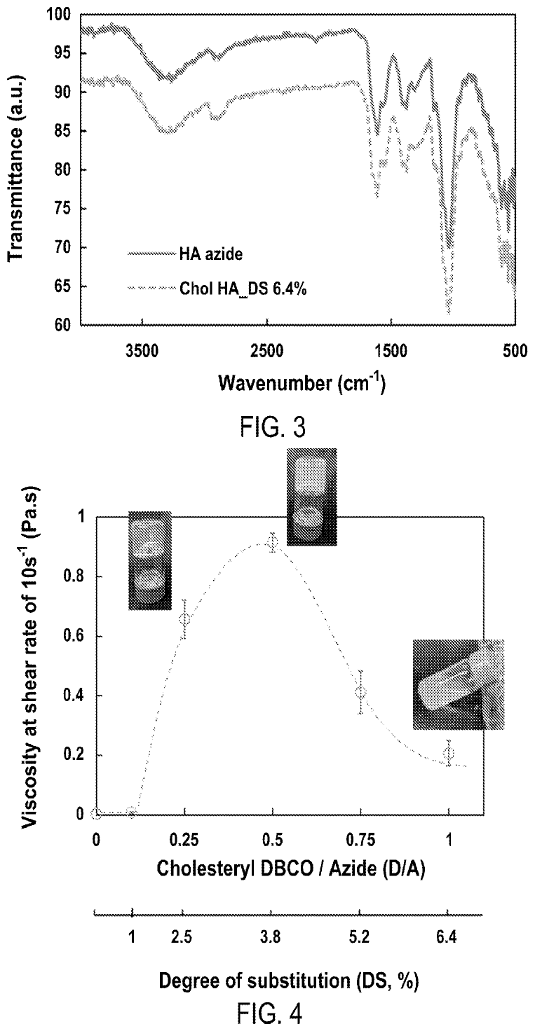

The DS of cholesterol increased when D/A increased although the reaction efficiency decreased when D/A was above 0.25, likely resulting from steric hindrance of the bulky cholesterol substituents (FIG. 2). The successful coupling via click chemistry was also validated by FTIR where the peak (˜2200 cm⁻¹) ascribable to the azide group on HA azide nearly disappeared after reacting with sufficient amount of cholesterol DBCO (D/A=1) (FIG. 3).

### Synthesis of 4-Arm PEG20k-DBCO

4-arm PEG20k-amine (100 mg, 0.02 mmol) was dried at 90° C. in vacuum oven for 30 min and stored in a desiccator before the use. Dried 4-arm PEG20k amine was dissolved in 3 mL of dry dichloromethane and mixed with triethylamine (11 μL, 0.08 mmol), to which DBCO NHS ester (12.1 mg, 0.03 mmol), dissolved in 2 mL of dry dichloromethane, was added. The reaction was carried out for 4 hr at RT in argon atmosphere. After the solvent was removed, flash column chromatography was carried out to purify the product with chloroform/methanol 4:1 as an eluent. The product was concentrated to −5 mL and precipitated in the cold ether (100 mL). Filtration was performed with a nylon membrane (0.45 μm) to obtain white solids. Residual ether was removed in a vacuum oven at RT overnight. Dried 4-arm PEG-20k-DBCO was stored at −20° C. before use. Synthesis was confirmed by ¹H NMR (CDCl₃).

### Aqueous Chol HA Formulations: Physical Gelation, Nanoparticle Formation, and Rheology

Chol HA synthesized in different D/A ratios were dissolved in PBS (pH 7.4) into 1% (w/w) solutions. Prolonged dissolution was required for Chol HA with D/A 0.75 and 1.0 ratios due to the higher amount of hydrophobic cholesterol introduced. Chol HA solutions were further dispersed using probe sonication (VCX130A, SONICS) in an ice bath (10s, repeated 3 times). Sonicated Chol HA solutions were stored at 4° C. overnight before use.

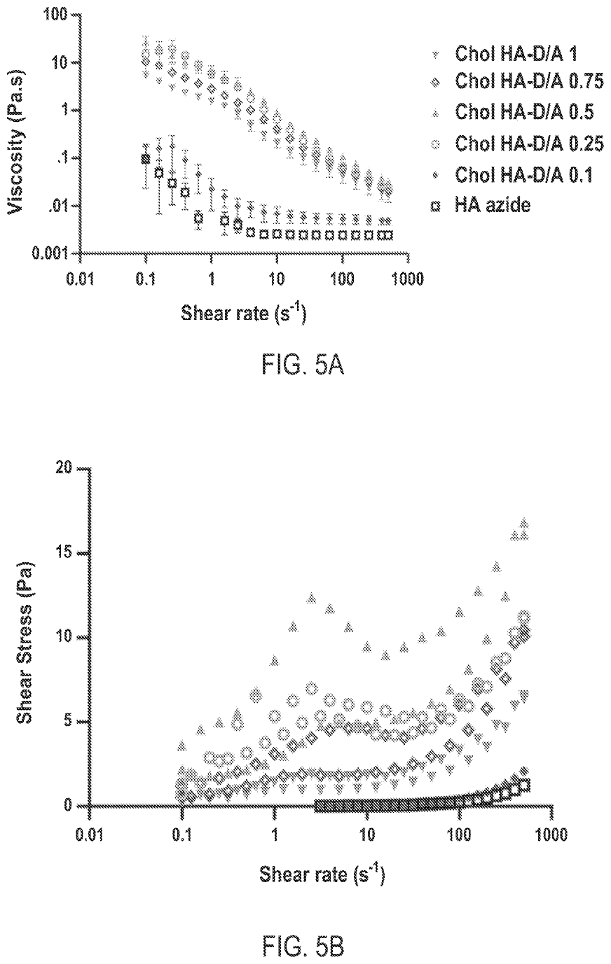

Steady shear viscosities of Chol HA solutions were measured using a rheometer (AR 2000ex, TA instruments), equipped with a cone-and-plate geometry (2°, 40 mm). Steady shear viscosities were recorded from a shear rate of 0.1 to 500 s⁻¹ and then 500 to 0.1 s⁻¹. The intermolecular hydrophobic cholesterol-cholesterol interactions in Chol HA translated into physical gellation that can be readily virtualized by flipping the vial (FIG. 4 insets), which were also consistent with their measured viscosities at 10 s⁻¹ that significantly increased as D/A increased from 0.1 to 0.5 (FIG. 4). All Chol HA formulations examined exhibited shear thinning behavior (FIG. 5A). Thixotropy, a type of fluid behavior representing time-dependent reversible viscosity, was observed in Chol HA solutions with D/A >0.1 (FIG. 5B). However, as D/A ratio was further increased >0.5, the viscosity of Chol HA at 10 s⁻¹ decreased (FIG. 4).

As depicted in FIG. 11, these results suggest that 2.5-3.8% DS, the increasing hydrophobic interactions (Chol-Chol) in Chol HA enabled interchain physical crosslinking, facilitating their gelation. As DS of cholesterol further increased (>3.8%), the intrachain Chol-Chol hydrophobic clustering resulted in the formation of Chol HA nanoparticles and the reduction of viscosity of the NP suspension.

### Chemical Crosslinking of Chol HA Hydrogels and Rheology

Chol HA hydrogels were prepared by mixing aqueous 4-armPEG20k-DBCO and sonicated Chol HA solutions with molar ratios of [DBCO]:[available N₃] from 1 to 0 in PBS (pH 7.4) by gently mixing them with a pipet and transferred into Teflon molds (6.0 mm diameter, 80 μL) and allowed to gel at RT. HA azide hydrogel (0 DBCO) was also prepared with 4-armPEG-DBCO as a control. Gelling time varied among different hydrogel formulations and was determined by rheology.

To investigate the shear moduli over the course of chemical crosslinking/gelling, a parallel plate (20 mm) was used (AR 2000ex, TA instruments). Immediately after mixing Chol HA and 4-arm PEG20k DBCO in a microfuge tube, the mixture (˜0.32 mL) was loaded onto the rheometer and the measuring gap of 1 mm was set. Oscillatory measurements were immediately carried out within the linear viscoelastic region with strain of 1% and frequency of 1 Hz.

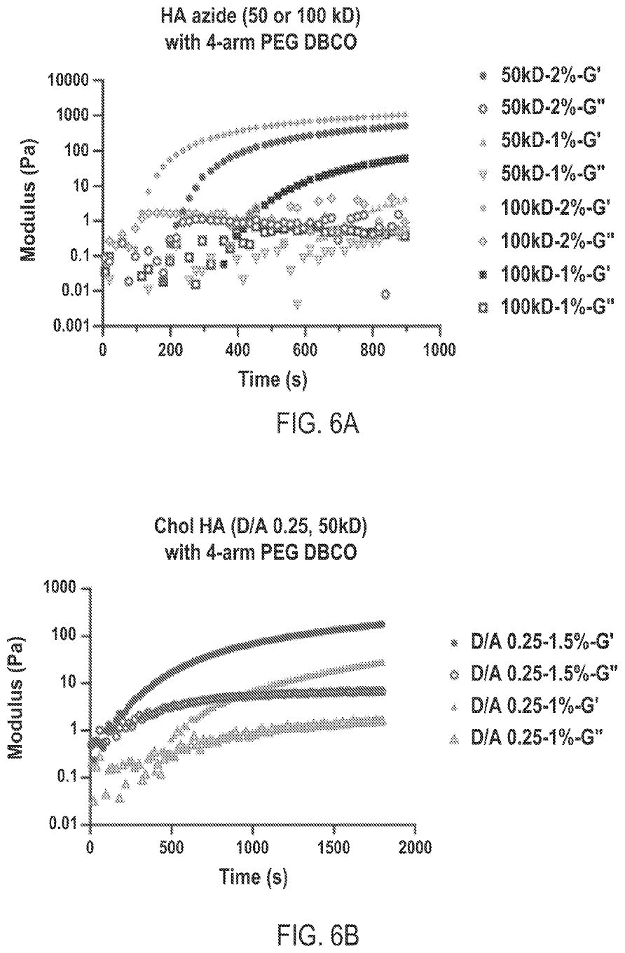

As shown in FIG. 6A-6B, HA azide (1 and 2%) with Mw of 50 and 100 kD were evaluated. Only the 2% of HA azide formulation formed a strong and free-standing gel upon chemical crosslinking with 4-arm PEG20k-DBCO (FIG. 6A). The 100-kD HA azide (2%) gelled faster with 4-arm PEG20k-DBCO (gelling time of 5-6 min) than the 50-kD HA azide (gelling time 9-10 min). After incorporation of cholesterol to HA azide, 1.5% of Chol HA (D/A 0.25) was able to form a free-standing gel after chemical crosslinking with 4-arm PEG20k-DBCO but with a longer gelling time (˜20 min) (FIG. 6B).

### Loading a Diverse Range of Hydrophobic and Hydrophilic Cargos in Chol HA Nanoparticles (NPs) or Hydrogels (Physically or Chemically Crosslinked): Incorporation of Nile Red or Dexamethasone in Chol HA NPs

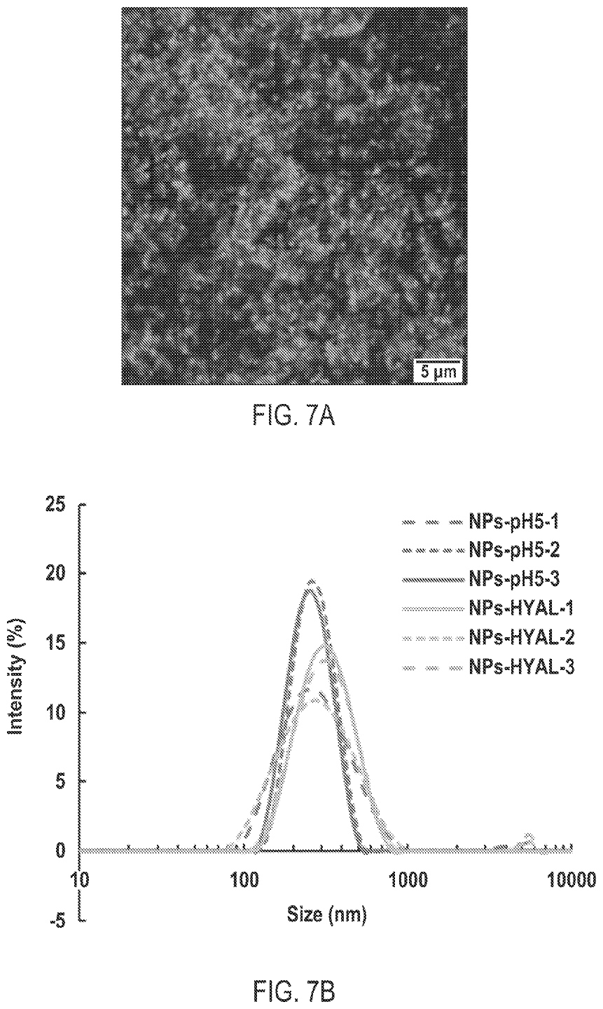

To visualize the Chol HA NPs, a hydrophobic dye Nile red was incorporated by sonicating acetone solution of Nile red (10 μL, 1 mg/mL) with aqueous Chol HA solution (1 mL, 2.5 mg/mL) to achieve a final Nile red concentration of 10 μg/mL of Chol HA solution. After overnight shaking in the dark, the Nile red-loaded Chol HA was observed with confocal laser scanning microscopy (CLSM; Excitation: 561 nm/Emission: 633 nm). Particle size distribution of Chol HA NPs was examined by dynamic light scattering (DLS) using a Zetasizer (Malvern Instruments). FIG. 7A revealed the morphologies of Nile red-loaded NPs in aqueous Chol HA (D/A=1). The diameter measured was around 400 nm, which approximated those determined by the DLS (FIG. 7B). The stability of Chol HA NPs was demonstrated by their resistance to enzymatic digestion. DLS of the Chol HA NPs before and after enzyme treatment showed minimal size changes after 4-hr incubation with hyaluronidase (FIG. 7B).

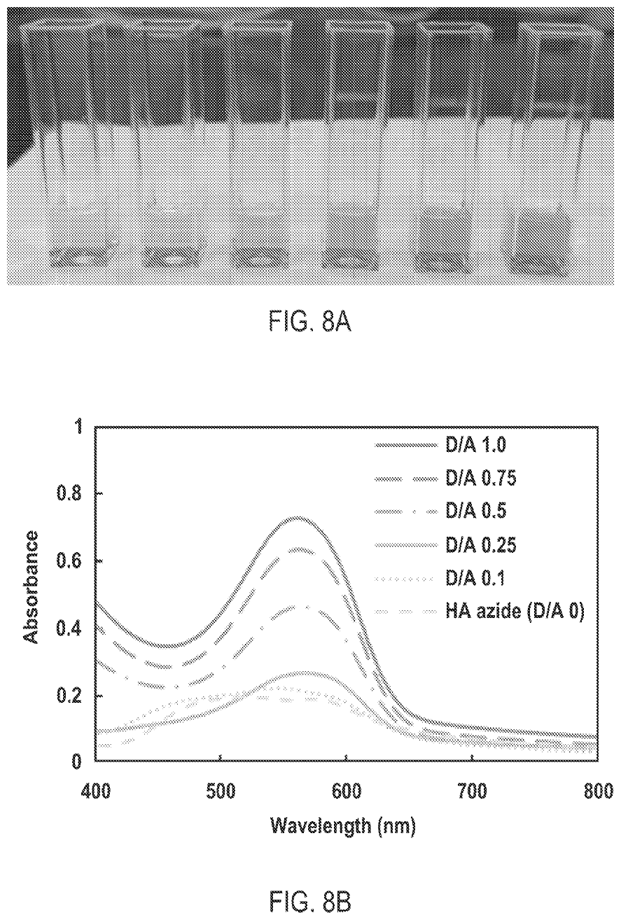

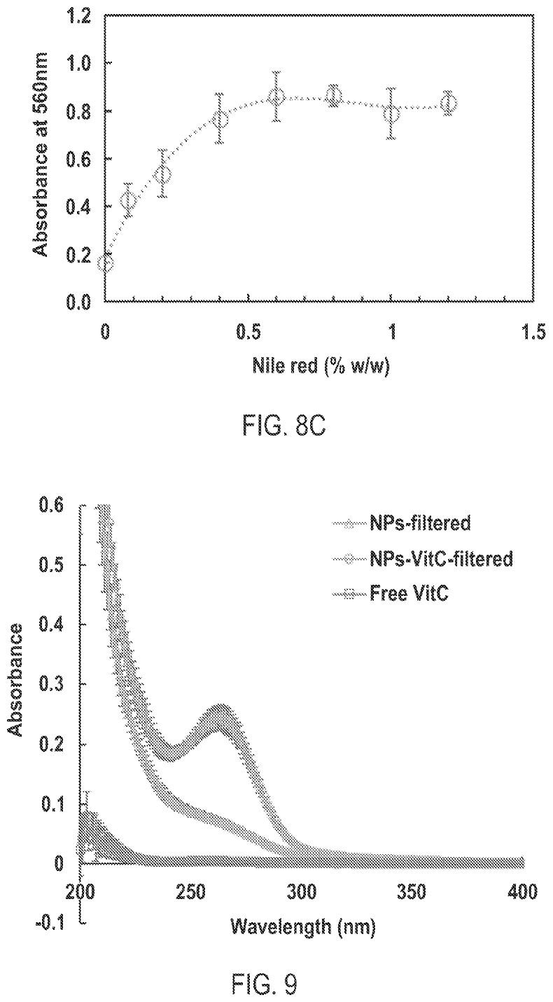

To evaluate the loading capacity of hydrophobic cargos by Chol HA NPs, Nile red and dexamethasone were each dissolved in acetone and ethanol, respectively, to prepare a stock solution of 1 mg/mL. Varying volumes of the stock solutions were added in aqueous Chol HA solutions while stirring to achieve a series of working solutions, which were subjected to probe sonication 3 times (10 s/time) in an ice bath, followed by overnight shaking in the dark at RT. The amount of Nile red loaded was determined by UV-Vis (FIG. 8C) while the amount of dexamethasone loaded was determined by a dexamethasone ELISA kit (CD Creative Diagnostics, which measures those not loaded).

Without being properly “dissolved” within the hydrophobic domain of Chol HA, Nile red would have precipitated out of aqueous solution and would not contribute to the solution absorbance. FIGS. 8A-8B showed that the Nile red incorporated within the hydrophobic domains enabled the visualization of the amount of hydrophobic domains in Chol HA as a function of D/A (positively correlated with the purple gradient). The loading capacity of Nile red in Chol HA (D/A=1) was determined to be ˜0.4% (w/w, relative to Chol HA) (FIG. 8C) while the loading capacity of dexamethasone, determined from ELISA, was about 0.08% (w/w, relative to Chol HA).

### Stabilization of Vitamin C by Chol HA NPs

The interaction of Chol HA with hydrophilic cargos was also evaluated. Vitamin C (VitC, i.e., ascorbic acid), a water-soluble vitamin, with a rapid decomposition rate in water, was also chosen to be protected by Chol HA NPs. Without the need of co-sonication, different amount of VitC stock solutions was directly added into sonicated Chol HA solutions, followed by overnight shaking in dark. The amount of VitC not decomposed after incubation was measured by UV-Vis at the characteristic 265 nm adsorption (FIG. 9), which revealed that the Chol HA significantly enhanced the stability of Vitamin C (presumably via interaction with its hydrophilic shell).

### In Vitro Cellular Uptake of Chol HA NPs

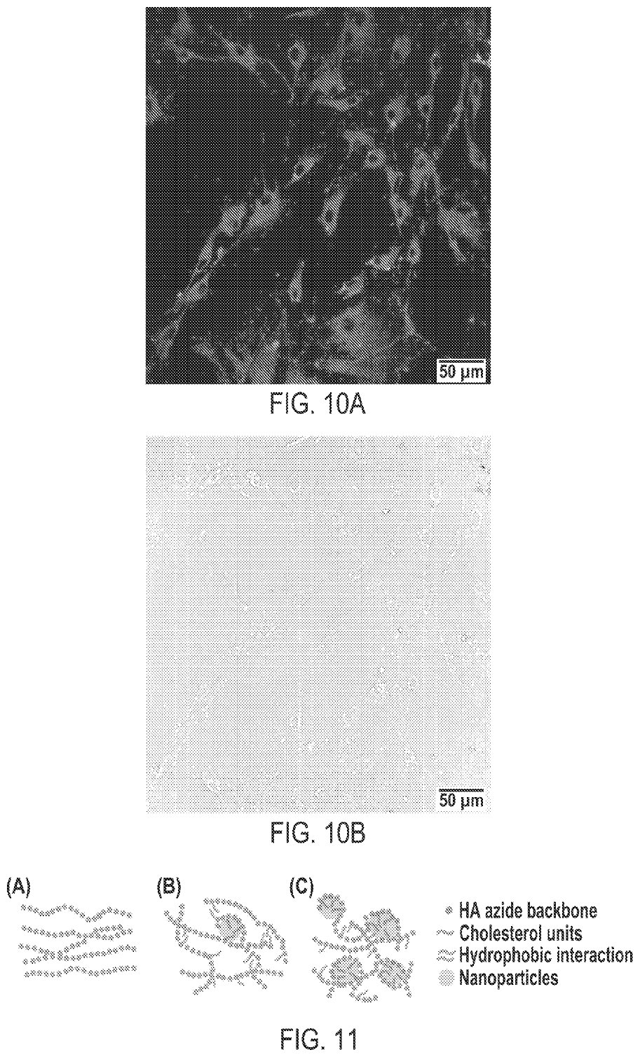

Rat bone marrow stromal cells (MSCs) were seeded onto poly-L-lysine coated coverslips (5000 cells/cm²) and cultured for two days prior to the addition of Nile red (NR)-loaded Chol HA NPs (250 μg/mL). The MSCs were exposed to NPs for different periods of time, and the un-internalized NPs were removed by washing the cells three times with PBS prior to microscopy. The fluorescence microscopy images were taken with CLSM (Excitation: 561 nm/Emission: 633 nm). We showed that after 4-hr incubation with Chol HA NPs, a large amount of NPs were uptaken by the MSCs, supporting excellent cell membrane permeability of these NPs (FIGS. 10A-10B).

### Coupling of Oligonucleotides to HA Derivatives Via Click Chemistry

Following an EDC/NHS amidation procedure, hyaluronic acid (HA) was dissolved in 50% DMSO aqueous solution, to which EDC was added, followed by NHS. The mixture was stirred for 10 min before DMSO solution of DBCO amine was added. The reaction was carried out at RT with vigorous stirring. Concentrated NaCl was introduced to screen out the charge of HA before precipitation with ethanol. The precipitate was obtained after centrifugation (2000 rpm, 5 min) and redissolved in DI water for dialysis. Cotton-like HA DBCO was obtained after lyophilization. Oligonucleotide 5′Carboxy-mCmGTTmCmG-3′Azide (Integrated DNA Technologies) was coupled to HA DBCO via copper-free, strain-promoted azide-alkyne click chemistry in water with a pre-determined stoichiometric ratio. Successful conjugation of the oligonucleotide was confirmed by ¹H NMR.

### Cell Encapsulation in Chol HA Hydrogels

To encapsulate cells in a Chol HA hydrogel, a 25 μL suspension of rat bone marrow stromal cells (MSCs) of varying numbers was mixed with Chol HA and 4-arm PEG20k DBCO in a pre-determined stoichiometric ratio in PBS and pipetted onto sterilized Parafilm and allowed to gel for 10-20 min before being transferred into low-attachment 24-well culture plates. Each cell-laden hydrogel was cultured in 1 mL MSC expansion media (DMEM-LG, 20% FBS, 1% Penn-Strep), and the metabolic activity of encapsulated cells was determined using the CCK8 (Dojindo, Japan) kit as per the vendor instructions after 24 h, 3, 5 and 7 days.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A modified hyaluronic acid comprising one or more covalently modified monomers defined by Formula I 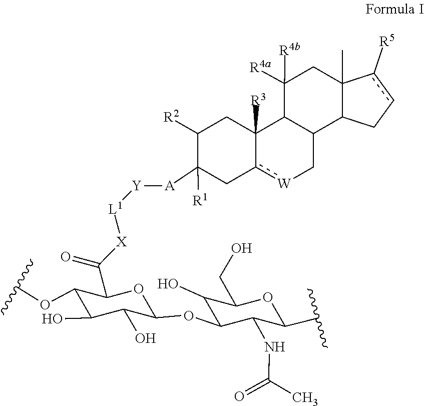
wherein [symbol]
denotes carbon-carbon bond or carbon-carbon double bond;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two click motifs;
X is NH, O, or S;
R^{a} is 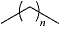
wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R⁴ and Rb are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is 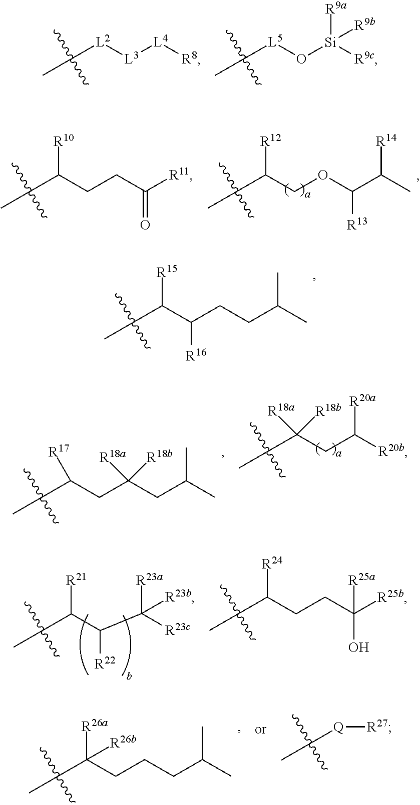
L² is absent, 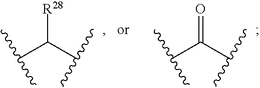
R²⁸ is absent or C₁-C₆ alkyl;
L³ is absent, or 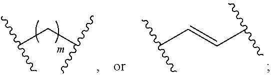
m is an integer 1, 2, or 3;
L⁴ is absent 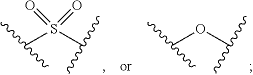
R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or 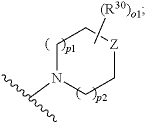
R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form 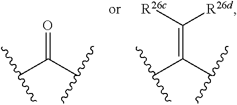
were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

2. The modified hyaluronic acid of claim 1, wherein the modified hyaluronic acid comprises a random copolymer defined by Formula II below 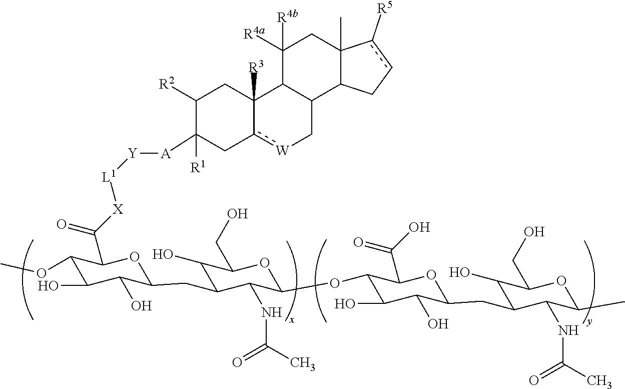
Formula II
wherein
x and y are each independently integers from 1 to 2500, wherein x+y is no more than 2500, and wherein x and y represent the relative portion of each monomer within the random copolymer; [symbol]
denotes carbon-carbon bond or carbon-carbon double bond;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is NH, O, or S;
R^{a} is 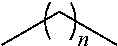
wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is 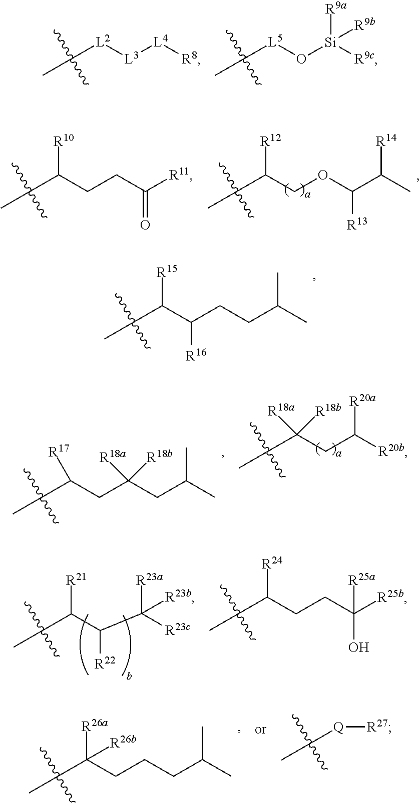
L² is absent, 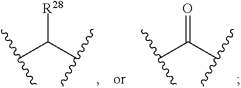
R²⁸ is absent or C₁-C₆ alkyl;
L³ is absent, or 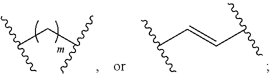
m is an integer 1, 2, or 3;
L⁴ is absent, 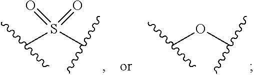
R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or 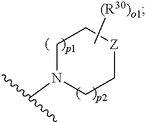
R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form 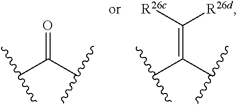
were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{29b} are each independently C₁-C₆ alkyl, C₁-C₆ heteroalkyl, halogen, or hydroxyl;
Q is O, S, or NR⁶; and
R²⁷ is C₁-C₆ alkyl.

3. The modified hyaluronic acid of any of claims 1-2, wherein the modified hyaluronic acid is covalently crosslinked.

4. The modified hyaluronic acid of claim 3, wherein the modified hyaluronic acid is covalently crosslinked using click chemistry.

5. The modified hyaluronic acid of any of claims 1-2, wherein the modified hyaluronic acid comprises a random copolymer defined by Formula III below 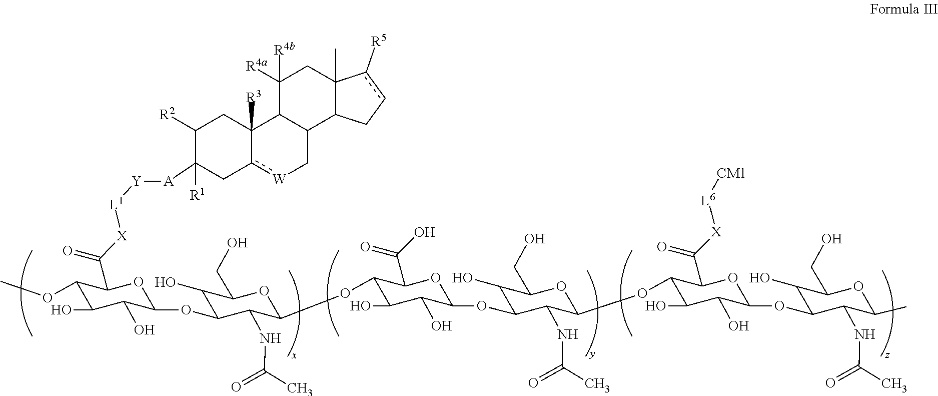
wherein
x, y, and z are each independently integers from 1 to 2500, wherein x, y, and z represent the relative portion of each monomer within the random copolymer, and wherein x+y+z is no more than 2500; [symbol]
denotes carbon-carbon bond or carbon-carbon double bond;
L⁶ is absent, or represents a linking group;
CM1 represents a first click motif;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is, independently for each occurrence, NH, O, or S;
R^{a} is 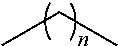
wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR^{4a}; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is 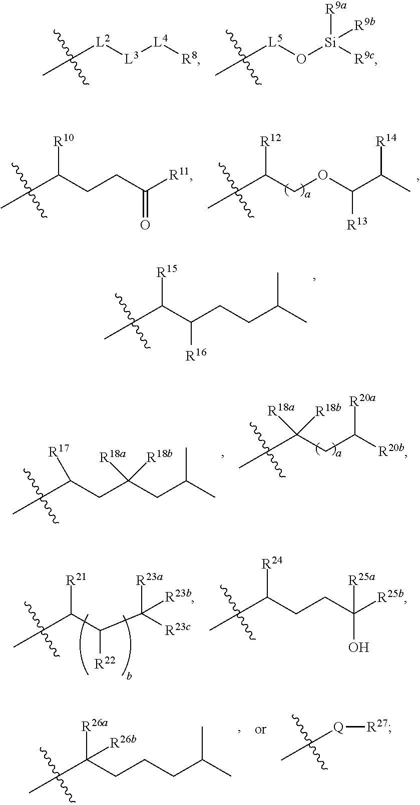
L² is absent, 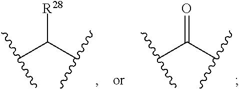
R²⁸ is absent or C₁-C₆ alkyl;
L³ is absent, or 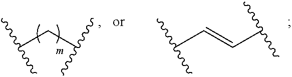
m is an integer 1, 2, or 3;
L⁴ is absent, or 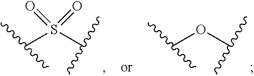
R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or 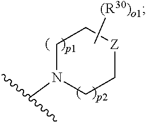
R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form 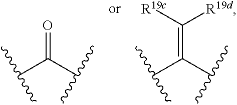
were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

6. The modified hyaluronic acid of claim 5, wherein CM1 comprises an azide.

7. The modified hyaluronic acid of any of claims 5-6, wherein the modified hyaluronic acid is covalently crosslinked by reaction of the copolymer defined by Formula III with a crosslinker defined by the structure below 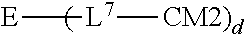
wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

8. The modified hyaluronic acid claim 7, wherein CM2 comprises an alkyne.

9. The modified hyaluronic acid of claim 8, wherein the CM2 comprises a dibenzocyclooctyne (DBCO) moiety.

10. The modified hyaluronic acid of any of claims 7-9, wherein the click reaction comprises a strain-promoted alkyne-azide cycloaddition (SPAAC).

11. The modified hyaluronic acid of any of claims 7-10, wherein E comprises an oligomer or polymer.

12. The modified hyaluronic acid of any of claims 1-11, wherein R⁵ is 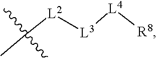
wherein
L² is absent, 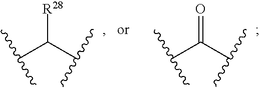
R²⁸ is absent or C₁-C₆ alkyl;
L³ is absent, or 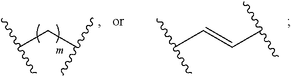
m is an integer 1, 2, or 3;
L⁴ is absent, 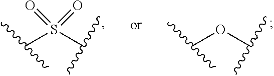
and
R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl.

13. The modified hyaluronic acid of any one of claims 1-13, wherein R¹ is —CH₃.

14. The modified hyaluronic acid of any one of claims 1-14, wherein A is O.

15. The modified hyaluronic acid of any one of claims 1-15, wherein L² is

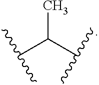 [SMILES: *C(*)C]

16. The modified hyaluronic acid of any one of claims 1-15, wherein L³ is

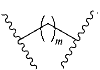

17. The modified hyaluronic acid of any one of claims 1-16, wherein m is 3.

18. The modified hyaluronic acid of any one of claims 1-17, wherein L⁴ is absent.

19. The modified hyaluronic acid of any one of claims 1-18, wherein R⁸ is methyl, ethyl, n-propyl, isopropyl, n-butyl, isobutyl, t-butyl, pentyl, hexyl, heptyl, octyl, nonyl, or decyl.

20. The modified hyaluronic acid of any one of claims 1-19, wherein R⁸ is isopropyl.

21. The modified hyaluronic acid of any one of claims 1-20, wherein the modified hyaluronic acid comprises one or more covalently modified monomers defined by Formula IA 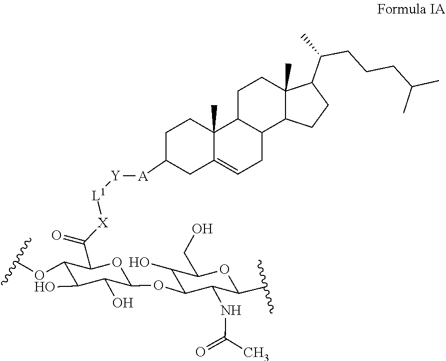
wherein
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two click motifs;
X is NH, O, or S; and
R^{a} is 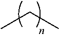
wherein n is an integer from 0 to 12.

22. The modified hyaluronic acid of any of claims 1-21, wherein the modified hyaluronic acid comprises a random copolymer defined by Formula TIA below 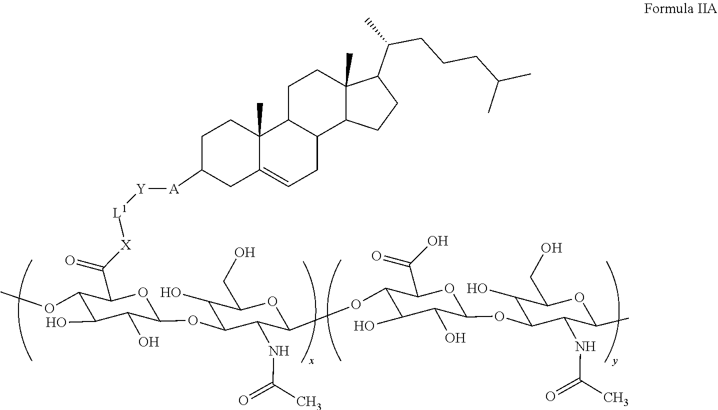
wherein
x and y are each independently integers from 1 to 2500, wherein x and y represent the relative portion of each monomer within the random copolymer, and wherein x+y+z is no greater than 2500;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two dick motifs;
X is NH, O, or S; and
R^{a} is 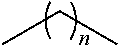
wherein n is an integer from 0 to 12.

23. The modified hyaluronic acid of any of claims 1-21, wherein the modified hyaluronic acid comprises a random copolymer defined by Formula IIIA below 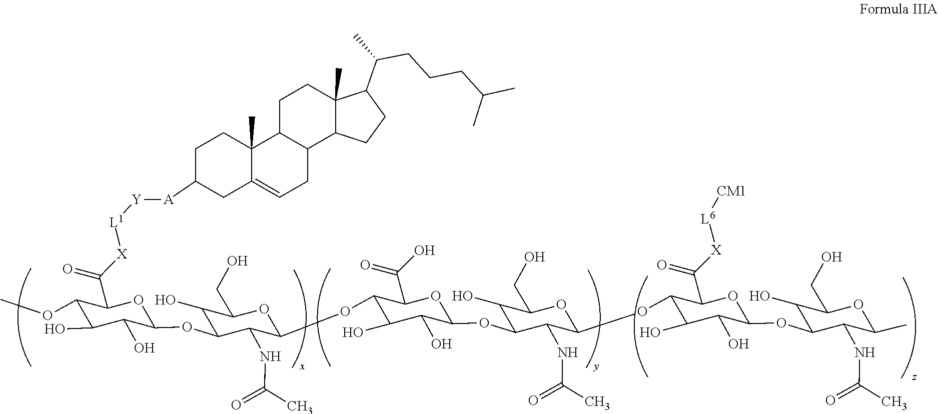
wherein
x, y, and z are each independently integers from 1 to 2500, wherein x, y, and z represent the relative portion of each monomer within the random copolymer, and wherein x+y+z is no greater than 2500;
L⁶ is absent, or represents a linking group;
CM1 represents a first click motif;
A is O or S;
Y is —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is a linking group comprising a moiety formed by the chemical reaction of two click motifs;
X is, independently for each occurrence, NH, O, or S;
R^{a} is 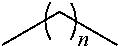
wherein n is an integer from 0 to 12.

24. The modified hyaluronic acid of claim 23, wherein CM1 comprises an azide.

25. The modified hyaluronic acid of any of claims 23-24, wherein the modified hyaluronic acid is covalently crosslinked by reaction of the copolymer defined by Formula IIIA with a crosslinker defined by the structure below 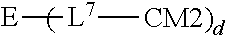
wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

26. The modified hyaluronic acid claim 25, wherein CM2 comprises an alkyne.

27. The modified hyaluronic acid of claim 26, wherein the CM2 comprises a dibenzocyclooctyne (DBCO) moiety.

28. The modified hyaluronic acid of any of claims 25-27, wherein the click reaction comprises a strain-promoted alkyne-azide cycloaddition (SPAAC).

29. The modified hyaluronic acid of any of claims 25-28, wherein E comprises an oligomer or polymer.

30. The modified hyaluronic acid of any one of claims 1-29, wherein X is NH.

31. The modified hyaluronic acid of any one of claims 1-30, wherein Y is —NR^{a}C(═O)—.

32. The modified hyaluronic acid of any one of claims 1-31, L¹ comprises a moiety formed by a strain-promoted alkyne-azide cycloaddition (SPAAC).

33. The modified hyaluronic acid of any one of claims 1-32, L¹ comprises a moiety formed by reaction of an azide moiety with a dibenzocyclooctyne (DBCO) moiety.

34. The modified hyaluronic acid of any one of claims 1-33, wherein L¹ further comprises an oligomer or polymer.

35. A modified hyaluronic acid comprising one or more covalently modified monomers defined by Formula IV 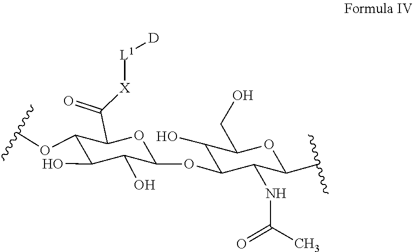
wherein
L¹ is a linking group comprising a moiety formed by the chemical reaction of two click motifs;
X is, independently for each occurrence, NH, O, or S;
D represents an oligomer or copolymer (block copolymer or random copolymer) defined by the formula below 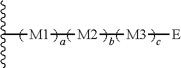
wherein M1 comprises a repeat unit comprising a hydrophilic sidechain;
wherein M2 comprises a repeat unit comprises a cholesterol-containing sidechain;
wherein M3 comprises a repeat unit comprising a hydrophilic sidechain;
a is an integer from 0 to 500;
is b is an integer from 2 to 500;
c is an integer from 0 to 500; and
E is absent, or represents a terminal unit optionally selected from the group consisting of dithioester, trithiocarbonate, xanthate.

36. The modified hyaluronic acid of claim 35, wherein the M1, M2, and M3 are independently repeat units obtainable by polymerization of a (meth)acrylate or (meth)acrylamide monomer, such as reversible addition-fragmentation chain-transfer polymerization (RAFT) of a (meth)acrylate or (meth)acrylamide monomer.

37. The modified hyaluronic acid of any of claims 35-36, wherein at least one of a and c is from 2 to 500.

38. The modified hyaluronic acid of any of claims 35-37, wherein M1 and M3 are independently repeat units obtainable by polymerization of a (meth)acrylate or (meth)acrylamide monomer comprising a hydrophilic oligomer or polymer sidechain (e.g., an oligo- or polyethylene glycol sidechain), such as reversible addition-fragmentation chain-transfer polymerization (RAFT) of a (meth)acrylate or (meth)acrylamide monomer comprising a hydrophilic oligomer or polymer sidechain, such as an oligo- or polyethylene glycol sidechain.

39. The modified hyaluronic acid of any of claims 35-38, wherein M2 is a repeat unit obtainable by polymerization of a (meth)acrylate or (meth)acrylamide monomer comprising a (meth)acrylate or (meth)acrylamide monomer comprising a sidechain defined by the formula below, such as reversible addition-fragmentation chain-transfer polymerization (RAFT) of a (meth)acrylate or (meth)acrylamide monomer comprising a sidechain defined by the formula below 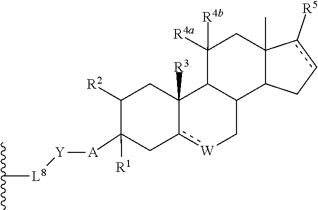
A is absent, or is O or S;
Y is absent, or represents —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; [symbol]
denotes carbon-carbon bond or carbon-carbon double bond;
L⁸ is absent, or is a linking group;
R^{a} is 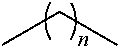
wherein n is an integer from 0 to 12;
R¹ is H, C₁-C₆ alkyl, C₂-C₆ alkenyl, or C₂-C₆ alkynyl;
R² is H or OR⁶;
R³ is H or —CH₃;
R^{4a} and R^{4b} are each independently H or OR⁶, or R^{4a} and R^{4b}, together with the atom to which each is attached, combine to form a cycloalkyl, aryl, heterocycloalkyl, or heteroaryl ring;
W is CR^{4a} or CR^{4a}R^{4b}, where if a double bond is present between W and the adjacent carbon, then W is CR⁴a; and if a single bond is present between W and the adjacent carbon, then W is CR^{7a}R^{7b};
R⁶ is H or C₁-C₆ alkyl;
R^{7a} and R^{7b} are each independently H, halogen, or C₁-C₆ alkyl;
R⁵ is 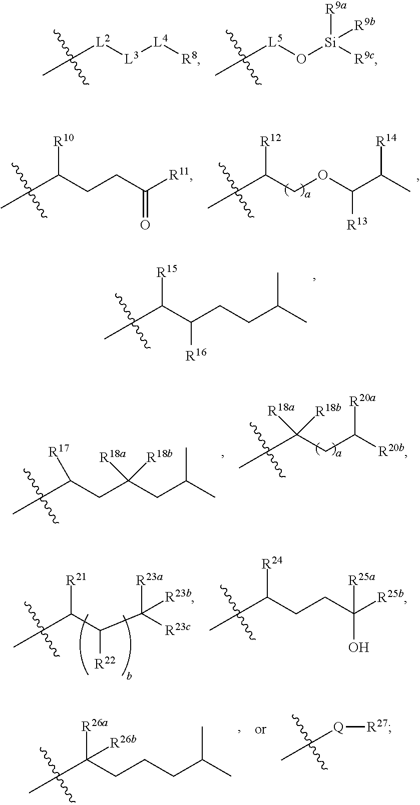
L² is absent, 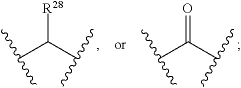
R²⁸ is absent or C₁-C₆ alkyl;
L³ is absent, or 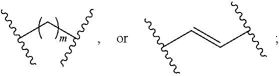
m is an integer 1, 2, or 3;
L⁴ is absent, 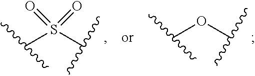
R⁸ is a C₃-C₁₀ alkyl, C₃-C₁₀ cycloalkyl, C₃-C₁₀ alkenyl, C₃-C₁₀ cycloalkenyl, C₃-C₁₀ alkynyl, C₃-C₁₀ aryl, C₂-C₉ heterocyclyl, or C₂-C₉ heteroaryl;
L⁵ is C₁-C₆ alkylene;
R^{9a}, R^{9b}, and R^{9c} are each independently C₁-C₆ alkyl or C₆-C₁₀ aryl;
R¹⁰ is H or C₁-C₆ alkyl;
R¹¹ is —OR⁶, —NR^{29a}R^{29b}, or 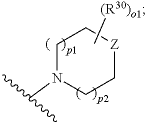
R^{29a} and R^{29b} are each independently H, —OR⁶, C₆-C₁₀ aryl, or C₁-C₆ alkyl;
R³⁰ are each independently halogen or C₁-C₆ alkyl;
o1 is an integer from 0 to 8;
p1 and p2 are each independently an integer from 0 to 2;
Z is CH₂, O, S, or NR⁶;
a is 0 or 1;
R¹² is H or C₁-C₆ alkyl;
R¹³ is C₁-C₆ alkyl;
R¹⁴ is H or C₁-C₆ alkyl;
R¹⁵ is H or C₁-C₆ alkyl;
R¹⁶ is halo, hydroxyl, C₁-C₆ alkyl, or C₁-C₆ heteroalkyl;
R¹⁷ is H or C₁-C₆ alkyl;
R^{18a} and R^{18b} are each independently C₁-C₆ alkyl;
R^{19a} and R^{19b} are each independently H, C₁-C₆ alkyl, or R^{26a} and R^{19b}, together with the atom to which each is attached combine to form 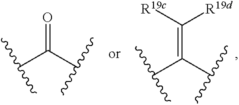
were R^{19c} and R^{19d} are each independently H or substituted C₁-C₆ alkyl;
R^{20a} and R^{20b} are each independently H, hydroxyl, or C₁-C₆ alkyl;
R²¹ is H or C₁-C₆ alkyl;
R²² is H or C₁-C₆ alkyl;
R^{23a}, R^{23b}, and R^{23c} are each independently C₁-C₆ alkyl;
b is 1, 2, or 3;
R²⁴ is H or C₁-C₆ alkyl;
R^{25a} and R^{25b} are each independently C₁-C₆ alkyl;
R^{26a} and R^{26b} are each independently C₁-C₆ alkyl, C₁-C₆ heteroalkyl, halogen, or hydroxyl;
Q is O, S, or NR⁶; and
R²⁷ is C₁-C₆ alkyl.

40. The modified hyaluronic acid of any of claims 35-38, wherein M2 is a repeat unit obtainable by polymerization of a (meth)acrylate or (meth)acrylamide monomer comprising a (meth)acrylate or (meth)acrylamide monomer comprising a sidechain defined by the formula below, such as reversible addition-fragmentation chain-transfer polymerization (RAFT) of a (meth)acrylate or (meth)acrylamide monomer comprising a sidechain defined by the formula below 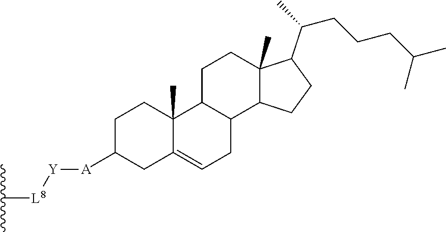
A is absent, or is O or S;
Y is absent, or represents —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; and
L⁸ is absent, or is a linking group.

41. The modified hyaluronic acid of any of claims 35-40, wherein the modified hyaluronic acid comprises a random copolymer defined by Formula IVA below 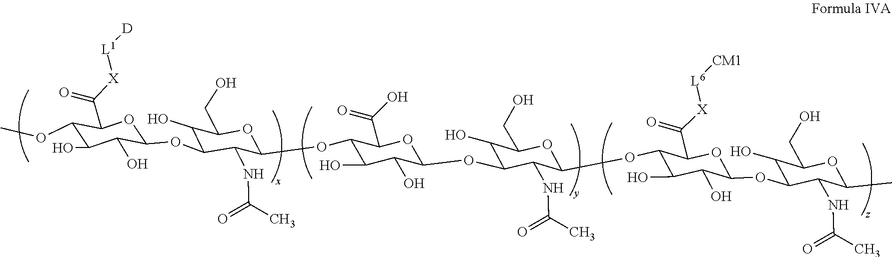
wherein
x, y, and z are each independently integers from 1 to 2500, wherein x, y, and z represent the relative portion of each monomer within the random copolymer, and wherein x+y+z is no greater than 2500;
L¹ is a linking group comprising a moiety formed by the chemical reaction of two click motifs;
X is, independently for each occurrence, NH, O, or S;
D represents an oligomer or copolymer (block copolymer or random copolymer) defined by the formula below 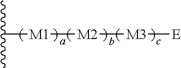
wherein M1 comprises a repeat unit comprising a hydrophilic sidechain;
wherein M2 comprises a repeat unit comprises a cholesterol-containing sidechain;
wherein M3 comprises a repeat unit comprising a hydrophilic sidechain;
a is an integer from 0 to 500;
b is an integer from 2 to 500;
c is an integer from 0 to 500;
E is absent, or represents a terminal unit optionally selected from the group consisting of dithioester, trithiocarbonate, xanthate;
L⁶ is absent, or represents a linking group; and
CM1 represents a first click motif.

42. The modified hyaluronic acid of claim 41, wherein CM1 comprises an azide.

43. The modified hyaluronic acid of any of claims 41-42, wherein the modified hyaluronic acid is covalently crosslinked by reaction of the copolymer defined by Formula IVA with a crosslinker defined by the structure below 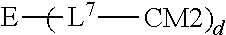
wherein
d represents an integer from 2 to 12, such as from 2 to 8, from 2 to 6, or from 2 to 4;
E represents a bivalent or polyvalent linking group,
L⁷ is absent or represents, individually for each occurrence, a bivalent linking group; and
CM2 represents a second click motif;
wherein the first click motif and the second click motif comprise a click motif pair that can participate in a click reaction to form one or more covalent bonds.

44. The modified hyaluronic acid claim 43, wherein CM2 comprises an alkyne.

45. The modified hyaluronic acid of claim 44, wherein the CM2 comprises a dibenzocyclooctyne (DBCO) moiety.

46. The modified hyaluronic acid of any of claims 43-45, wherein the click reaction comprises a strain-promoted alkyne-azide cycloaddition (SPAAC).

47. Nanoparticles or microparticles formed from the modified hyaluronic acid of any one of claims 1-46.

48. The nanoparticles or microparticles of claim 47, further comprising an active agent encapsulated within the nanoparticles or microparticles.

49. The nanoparticles or microparticles of claim 48, wherein the active agent comprises a hydrophobic small molecule therapeutic agent, a hydrophilic small molecule therapeutic agent, a macromolecule therapeutics agent, a diagnostic agent, a steroid, a vitamin, and oligonucleotide, an antibiotic, an antimicrobial peptide, an antimicrobial metal, a protein, a cell, a mineral, or a bioceramic.

50. A hydrogel comprising the modified hyaluronic acid of any one of claims 1-46.

51. The hydrogel of claim 51, further comprising an active agent dispersed in the hydrogel.

52. The hydrogel of claim 51, wherein the active agent comprises a hydrophobic small molecule therapeutic agent, a hydrophilic small molecule therapeutic agent, a macromolecule therapeutics agent, a diagnostic agent, a steroid, a vitamin, and oligonucleotide, an antibiotic, an antimicrobial peptide, an antimicrobial metal, a protein, a cell, a mineral, or a bioceramic.
